SEQUENCE LISTING (8 sequences)

SEQ ID NO: 1 - DNA, 104 bp, Oryza sativa
gcagcggcggcggcggcagctggacggcggcgttcatgccgagcccgacgggattcatgcacccgtggagcctcgccgcccggcagcagaaggccgccgccgag

SEQ ID NO: 2 - DNA, 284 bp, Oryza sativa
gtcagacctcgccgccgccggccgccaccgctgttccacctcacggacgtcatcacctccggcgccggcacgccccgcacggccgatgcgcggcggtggctgtcggagttcgccgagtcgctgcacctccccttccgctacacctccctccacgtccacgacggcgacgatgacgacgaactccatcacgagctcgccatgatctgcaacggcagctcatcaccaccggtgatcctcacatgcgacgacacgacgacgacgacgaacacgccattgcgcagcag

SEQ ID NO: 3 - DNA, 400 bp, Oryza sativa
gttcagcgatgccggcgagggcatcagcggcgtcgtctcgcggcggtgcccgagcctggattgcctcgatcgagctcgagcacatcgccggcgtggaggcgctcaccctccgcaccgactcgctcctgagcctccgcctcgtctcggtctctcgtctccaacggctcgacgtggaggccggaaacctccgcaggatgcgggtggagtactgcttcgacgaaacgagatcgtgtcgtccttggaacggcggcggcgcggcggcggtgatgcggctctccgcgccggcgttggaggagctcggttgggaggacgcgtgccccgacgaggttgagcggattaggttaccgacgtgcctcagggagctcgtcgtcagcgagcttccttctcatattatccgcag

SEQ ID NO: 4 - DNA, 576 bp, Oryza sativa
gtatggctcgatcttctgcttttctttttcttctttttttttttttaattattcgggaattgaagcctcatcctattgggggcatacgcgacttggcggagaggacaatggcgacgcatttcagagctaaggggggaagggatatgattttgaggctgtaattgcttgcgaagttctccttttggaagggatcggtcctagccattctgagctgctaaaaattttatttgcggtacggagcgtcttctgataagaggacagaacgattagggtctcccgtaatctgcttttaaatgcttgatggagtcctagaacctaaaattatggaattttctctaatgttaagctaggaatgagggtactggtttcttcgtagatggaaggcgctctacttgttgctgccggaactctgttgagatcttgtgcttaaatgcaggggtttaactggctgtgtaatcttaggcgtggagtgagttgactatgaagaattctgaacatgtctaggaatcataatttcgaattttgtagcttgtggtttatgcagcatctgatactattcagttcctcgttctgtag

SEQ ID NO: 5 - DNA, 659 bp, Oryza sativa
gtacgccgccggagacaggctcgccgccgccgcggccctcgacatgccatggttctccaagaagctgagctagctagctagtagctacagtgatcaagtgtagcataaaaatcttgtagcggagaatcatacacgctgatagagcgctcccagtcgtcctcgcgcgggcgaccgggggcgaaccctagcccccggccgccgccacacccctcctctccccggcctcgccgccgccggagcatgtcgccggaaaccgtgcggccgctgggatggctgcggcgaggctccaacctaggcagcgtggccgcctcggcgtgaaggggcggcgtggagcttcgggaaggctgcaatggcggcgaggcgaggggcggcggatccggcgcagccaccaccggattcggcgcccagcatcggccacgaggcgagtggcggctggatccggtgtctccgctgccggatctgagccgtggtggtgcgggagcacggcggatgtagcgacgccggtggcgcgacgaccgtggctggtcggcggtggtgcgggagcgtggcgtcggcggggagacacgacgtcggcggcaagcgctatggctgggggcgcgggcggccgcagcaggtggcgcgacggccgtggctgaccggcgggcggctgtggccggcag

SEQ ID NO: 6 - DNA, 1016 bp, Oryza sativa
gtcagctctctgtcttccttccttccttctttcttcctggttggaggattgatcaaatcctgcaaaggattaggttttcttgggattggtgggtggtagtcttgtcctccccccaaatcgtttttgatttgatttgatttgggttgggttggggttcatcgaatcaatcatttcaagaacagagttattctcggcggatttcttccaccgccgtcagttcttgtgtggattgtgtctcttccgttctggtgagtccgattcggtttctcctttgctttcaatcgatctctcccgctagtactgttcttgctcatcccgtctgatgctgatgagttggtttcttgcagtttcggtttcagaatctgcagttcgtcgtcggaccggttgtttcctgagtcccatctgaggataggtatcggctaatcgctttgatctgaatttcgattcatcgctgtagagaaatgcgccgaaaaaaaaaagaaaagaaatttctctcttttattttcttgctcttctcgtggtctcgagaaggataatgccgtagcgataaagtccatagcttgcaaggctggcatctcatgtgctgtcgctaactccatgggcgtgattgattgctctgccatccccaaaaaaataaaatcccttgataatccgtatggcattttcgtgtctgcacactgcacggttgatgggaaagtactaggtgttgctttcggaatctctctttcctaagattgcttctttgaccattctcgtagtaatcttgtctcatgcgggcagttttgctctcaagaaatgggttcatattgtcgcacaatttggccgtgtgcttagttgcaatttgcaatgcggattctttcgagtcaacgacgcggaatgagattaaaatttgagatctttctgacggttatttgtggtgtgcgatgagaaacgtgttgctcttggaattctttcatggatgattcgtcgtagtaggaggtttctttgtcgtgtgcattgatcgaatttctgtgcag

SEQ ID NO: 7 - DNA, 1714 bp, Oryza sativa
gtagagttgaaactgttgtgatgtaatgtagttcatcttcaactaaattgctgttgtgcactattacttttttttcgccgggaaggccgaaggaggccatccaaatatattaagaaggagtaaaagttacaagaaaaaaaaaacatcacaatgcaccaaggtgcatgacctcacagcacgcactaccacccaagcacaaaccgctgagggtgaagcctagcaccaaatgacccccgctaagaatggccgaacgccgctaggcctacggcaacaccacagagacaagactccgaaaacgaagccaccacggtggtcacgacatccaggatgccgccatcgcccatctaaaaaagatgtggttttcacccagagaagcttatcgagaaaggaagagggtaacccttgacaacgccccaaggaggttacgacgcccgaagacatagccgctgccggcccagtgaattaccggactaggcttttgcctgggaccctacaaccttgactgtagatcgccgtccaccaactcagaaccactacacagacaaatggcaacacgtagcttccaccacaccgcactgacgcccctccatcgaccgacttcatcgaaccacaccgcactgacgcccctccgtcagccgacatcaaccaccgcccctgagagctggcccaggacccctccgttccaccacccgccggcctcctcgccagctttggacaaaaggagaaccgggactggatggcattgctttggcagcctgagtttcccccgcttgacgagctgccgcctcaatccaacatagaaactccccacagagaaggggaggagctccaggaagggcgagggtgccgaccggcaacggagaagacgacccaccgccgccagctccctttacactgccatctgggcttgcgccgacgccagagacgcttctgctgctccggctccggcgcaacccaccggcgcccccgtcgcccggccgccgcaccccatctggccgcgccgcatagcccacccggctgaggagggcaccgcggcgtcaccctggctgcccggctgccgaggcctgcctgcgccatgggggctcgccgccgcactgccgagccgcgcgagcccgggctgcccggaccggccgcgcgccgagctgccgccgccgctcccagccgccgccgcctctaggcagccgccatcccagccgtcgagccaccgccaccgcgcccaggctgccgcaccgggccagccgcgccgtccctgccgccgctgccgccgcccccccagccacctcgcgccgccgttgtgccagatccaggcgtggggtggtcggatccggccttggaggcgccggatctgctgcctccccacaccgcgccgccatcgccacctccggccgcggtggagggtcttcgccggcgtcgtcgtcccgtccacgtcacgccggggggagtcgccgggaggaagaacctcgccgccgccttccctgctcgctgccggctttgccgccggcgagctccggcggcggcgaggcggtggggagggagtggaggcgtgggcggctagggttttttcgccccctgagccacccgcgcgagagcgacgcgagaggttgttttcaggttatcaatttagtctgtgcactattactggcttccatttggtgcactactagtgatttacatgtctacttattacctttactttcttttttctgtgtttcag

SEQ ID NO: 8 - DNA, 98 bp, Oryza sativa
gtaggcttcctaggtagggatcccatcccttcgattccctactccctcccccgattgatttgatttgatttgatttaattcgattgcctgcttttcag

(19) United States
(12) Patent Application Publication
(10) Pub. No.: US 2012/0096583 A1
(43) Pub. Date: Apr. 19, 2012

(54) METHODS AND COMPOSITIONS FOR ENHANCED GENE EXPRESSION THROUGH INTRON FREE ENERGY REDUCTION

(75) Inventors: Kasimalai Azhakanandam, Durham, NC (US); Thomas Z. McNeill, Frisco, TX (US)

(73) Assignee: Syngenta Participations AG

(21) Appl. No.: 13/233,282

(22) Filed: Sep. 15, 2011

Related U.S. Application Data
(60) Provisional application No. 61/393,614, filed on Oct. 15, 2010.

(51) Int. Cl. C12N 15/113; C12P 21/00; G06F 19/00; A01H 5/00; C07H 1/00; C07K 14/00; C12N 15/82

(52) U.S. Cl. 800/278; 530/350; 435/69.1; 435/468; 800/298; 536/25.3; 536/24.1; 702/19

(57) ABSTRACT

The present invention provides a method for selecting an intron for enhanced expression of a nucleotide sequence encoding a polypeptide, comprising: a) determining a mean free energy value per base pair of one or more introns; b) determining a mean IMEter score of the one or more introns of (a); and c) selecting an intron having a mean free energy value per base pair of below about −0.268 kcal/mol/bp and a mean IMEter score of at least about −0.034/bp, thereby selecting an intron for enhanced expression of a nucleotide sequence encoding the polypeptide, wherein expression of a nucleotide sequence operably associated with the selected intron is enhanced as compared to expression of a nucleotide sequence not operably associated with an intron having a mean free energy value per base pair of below about −0.268 kcal/mol/bp and a mean IMEter score of at least about −0.034/bp.

### STATEMENT OF PRIORITY

This application claims the benefit, under 35 U.S.C. §119(e), of U.S. Provisional Application Ser. No. 61/393,614, filed Oct. 15, 2010, the entire contents of which are incorporated by reference herein.

### FIELD OF THE INVENTION

The present invention provides methods related generally to the field of expression of nucleic acids. Specifically, the present invention provides methods for selecting an intron for enhanced expression of a nucleotide sequence.

### BACKGROUND OF THE INVENTION

Many factors affect expression of nucleic acids including chromatin structure, transcription efficiency, transcription factors, mRNA stability and regulatory factors such as promoters, enhancers and silencers. In addition, introns are known to affect expression levels in eukaryotes and in many cases may have a larger influence than, for instance, promoters. (Rose et al. Plant Cell 20:543-551 (2008)). Thus, for example, efficiently spliced introns boost expression more than 10-fold while others have little or no effect (Id.). The first intron of maize shrunken-1 gene has been shown to increase gene expression 1000 fold in maize protoplasts (Maas et al. Plant Mol. Biol. 16(2):199-207 (1990)) and the first intron of rice rubi3 enhanced gene expression about 3-fold in stable transgenic rice (Lu et al. Mol. Genet. Genom. 279(6):563-572 (2008)). The effect of introns on increasing nucleic acid expression is termed intron-mediated enhancement (IME) (Id.). Despite the significant influence of introns on the expression of nucleic acids, little is known about the mechanism IME. Further, only a few introns (mostly first introns) have been evaluated experimentally and are known to enhance gene expression in plants. Most of this work has been done in monocots.

Accordingly, the present invention addresses the previous shortcomings of the art by providing methods for selecting and modifying an intron for enhanced expression of a heterologous nucleotide sequence.

### SUMMARY OF THE INVENTION

One aspect of the present invention provides a method of selecting an intron for enhanced expression of a nucleotide sequence encoding a polypeptide, comprising: a) determining a mean free energy value per base pair of one or more introns; b) determining an IMEter score of the one or more introns of (a); and c) selecting an intron having a mean free energy value per base pair of below about −0.268 kcal/mol/base pair (bp) and a mean IMEter score of at least about −0.034/bp, thereby selecting an intron for enhanced expression of the nucleotide sequence encoding the polypeptide, wherein expression of the nucleotide sequence operably associated with the selected intron is enhanced as compared to expression of a nucleotide sequence not operably associated with an intron having a mean free energy value per base pair of below about −0.268 kcal/mol/bp and a mean IMEter score of at least about 40.

A further aspect of the invention provides a method of selecting an intron for enhanced expression of a nucleotide sequence encoding a polypeptide, comprising: a) determining a free energy value of one or more introns; b) determining an IMEter score of the one or more introns of (a); and c) selecting an intron having a free energy value of below about −31.51 kcal/mol and an IMEter score of at least about 40, thereby selecting an intron for enhanced expression of the nucleotide sequence encoding the polypeptide, wherein expression of the nucleotide sequence operably associated with the selected intron is enhanced as compared to expression of a nucleotide sequence not operably associated with an intron having a free energy value of below about −31.51 kcal/mol and an IMEter score of at least about 40.

The present invention further provides a method of producing a heterologous polypeptide in a plant, plant part and/or plant cell comprising: introducing into the plant, plant part and/or plant cell a heterologous nucleic acid molecule comprising: a) a heterologous intron having a mean free energy value per base pair below about −0.268 kcal/mol/bp and a mean IMEter score of at least about −0.034/bp; and b) a nucleotide sequence encoding the heterologous polypeptide, operably associated with the heterologous intron, under conditions whereby the nucleotide sequence is expressed in the plant, plant part and/or plant cell, thereby producing a heterologous polypeptide in the plant, plant part and/or plant cell.

An additional aspect of the invention provides a method of producing a heterologous polypeptide in a plant, plant part and/or plant cell comprising: introducing into the plant, plant part and/or plant cell a heterologous nucleic acid molecule comprising: a) a heterologous intron having a free energy value of below about −31.51 kcal/mol and an IMEter score of at least about 40; and b) a nucleotide sequence encoding the heterologous polypeptide, operably associated with the heterologous intron, under conditions whereby the nucleotide sequence is expressed in the plant, plant part and/or plant cell, thereby producing a heterologous polypeptide in the plant, plant part and/or plant cell.

In other aspects, the present invention provides a method of making a plant cell that produces a heterologous polypeptide comprising: introducing into the plant cell a nucleic acid comprising: a) a heterologous intron having a free energy value per base pair of below about −0.268 kcal/mol/bp and an IMEter score of at least about −0.034/bp; and b) a nucleotide sequence encoding the heterologous polypeptide, operably associated with the heterologous intron, under conditions whereby the nucleotide sequence is expressed in the plant cell, thereby making a plant cell that produces a heterologous polypeptide.

In further aspects, the present invention provides a method of making a plant cell that produces a heterologous polypeptide comprising: introducing into the plant cell a heterologous nucleic acid molecule comprising: a) a heterologous intron having a free energy value of below about −31.51 kcal/mol and an IMEter score of at least about 40; and b) a nucleotide sequence encoding the heterologous polypeptide, operably associated with the heterologous intron, under conditions whereby the nucleotide sequence is expressed in the plant cell, thereby making a plant cell that produces a heterologous polypeptide.

The present invention further provides a method of modifying the nucleotide sequence of an intron to increase expression of a nucleic acid molecule operably associated with said intron, comprising: mutating one or more nucleotides of the nucleotide sequence of the intron, wherein said mutation results in a decrease in the mean free energy value per base pair (e.g., to below about 0.268 kcal/mol/bp) and an increase in the mean IMEter score (e.g., to at least about −0.034/bp) of the intron as compared with the mean free energy value and mean IMEter score of the intron without said modifying.

In other embodiments of the invention a method is provided for modifying the nucleotide sequence of an intron to increase expression of a nucleic acid molecule operably associated with said intron, the method comprising: mutating one or more nucleotides of the nucleotide sequence of the intron, wherein said mutation results in a decrease in the free energy value (e.g., to below about −31.51 kcal/mol) and an increase in the IMEter score (e.g., to at least about 40) of the intron as compared with the free energy value and IMEter score of the intron without said modifying.

The methods of the present invention additionally comprise regenerating a plant from a transformed plant cell of the present invention.

The present invention further provides transgenic plants, plant parts, plant cells, and crops made by the methods of the invention.

Other and further objects, features and advantages would be apparent and more readily understood by reading the following specification and by reference to the accompanying drawings forming a part thereof, or any examples of the embodiments of the invention given for the purpose of the disclosure.

### BRIEF DESCRIPTION OF THE DRAWINGS

FIG. 1 is a flowchart illustrating operations according to some embodiments of the present invention.

FIG. 2 is a block diagram illustrating systems according to some embodiments of the invention.

FIG. 3 is a flowchart illustrating operations according to additional embodiments of the present invention.

FIG. 4 is a block diagram illustrating systems according to additional embodiments of the invention.

FIG. 5 shows an exemplary expression cassette of the present invention. The intron, iZmUbi361-01, is replaced with selected introns (See, e.g., Table 1). prZmUbi361-02=promoter; iZmUbi361-01=intron; cGUS11=GUS gene; tZmUbi361-01=the maize terminator (construct 18508).

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

The present invention is directed to the discovery that by determining the free energy value per base pair (or the free energy value) and the IMEter score of an intron, an intron can be selected that enhances the expression of a nucleotide sequence operably associated with the intron. Thus, some embodiments of the invention provide a method of selecting an intron for enhanced expression of a nucleotide sequence encoding a polypeptide, said nucleotide sequence being operably associated with said intron. Accordingly, in some embodiments of the present invention an intron can be selected that enhances the expression of a heterologous nucleotide sequence encoding a polypeptide, wherein expression of a heterologous nucleotide sequence operably associated with the selected intron is enhanced as compared to expression of a nucleotide sequence not operably associated with the intron (e.g., as a control).

Accordingly, one aspect of the present invention provides a method of selecting an intron for enhanced expression of a nucleotide sequence encoding a polypeptide, comprising: a) determining a mean free energy value per base pair of one or more introns; b) determining a mean IMEter score of the one or more introns of (a); and c) selecting an intron having a mean free energy value per base pair of below about −0.268 kcal/mol/bp and a mean IMEter score of at least about −0.034/bp, thereby selecting an intron for enhanced expression of the nucleotide sequence encoding the polypeptide, wherein expression of the nucleotide sequence operably associated with the selected intron is enhanced as compared to expression of a nucleotide sequence not operably associated with the selected intron having a mean free energy value per base pair of below about −0.268 kcal/mol/bp and a mean IMEter score of at least about −0.034/bp. A further aspect of the present invention provides a method of selecting an intron for enhanced expression of a heterologous nucleotide sequence encoding a polypeptide, comprising: a) determining a mean free energy value per base pair of one or more introns; b) determining a mean IMEter score of the one or more introns of (a); and c) selecting an intron having a mean free energy value per base pair of below about −0.268 kcal/mol/bp and a mean IMEter score of at least about −0.034/bp, thereby selecting an intron for enhanced expression of the heterologous nucleotide sequence encoding the polypeptide, wherein expression of the heterologous nucleotide sequence operably associated with the selected intron is enhanced as compared to expression of a nucleotide sequence not operably associated with the selected intron having a mean free energy value per base pair of below about −0.268 kcal/mol/bp and a mean IMEter score of at least about −0.034/bp. In some embodiments, only the mean free energy value per base pair of one or more introns is determined. In other embodiments, the mean free energy value per base pair of one or more introns and the mean IMEter score for the one or more introns is determined. In still other embodiments, when both the mean free energy and the mean IMEter score are determined, the intron is selected on the basis of the mean free energy value only. In other embodiments, the intron is selected on the basis of both the mean free energy value and the mean IMEter score. The present invention further encompasses the introns selected by any of the methods described herein.

As used herein, “intron” is a nucleotide sequence within a nucleotide sequence of an organism, wherein the intron is not translated into protein. Introns are commonly found in precursor mRNA in eukaryotes. In prokaryotes, introns have been found in tRNA and rRNA. Introns are transcribed to precursor mRNA (pre-mRNA) and other non-protein coding RNAs and subsequently removed (spliced out) when the pre-mRNA is processed to mature RNA. Introns have a conserved 5′ splice site (GT) and 3′ splice site (AG), which facilitate the splicing process in Eukaryotes. As long as these two splice sites are intact, an intron can be placed in an expression cassette, including in front of a coding sequence or in the coding sequence, thereby facilitating the splicing process according to the particular expression cassette design. It is further envisioned that the introns of the present invention can comprise introns having non-canonical splice sites. Thus, for example, the splice sites of an intron selected using the methods of the present invention may be modified (prior to, during and/or after selection) to comprise nucleotides at the splice site that are different than the conserved splice site nucleotides so that they can function with, for example, a modified splicing protein (e.g., a modified spliceosome).

Introns are operably associated with promoter and exon. They are embedded between exons and are spliced out during RNA processing. Some genes have more than one intron and some genes have no introns. While it is not known for certain how introns function to increase transcription efficiency, a number of hypotheses have been presented. For example, it has been suggested that introns may function by stabilizing transcription factors. They may act as physical barriers for transcription factors and passively separate regulatory and coding regions. A low free energy intron may move two exon strands closer to one another and thereby increase the efficiency of the splicosome. Certain introns may function as part of the binding apparatus for the transcription factor, thereby assisting in increasing the efficiency of transcription. It is further believed that introns, along with other regions in a gene, may act together to form an energetically favorable area for transcription.

It has been reported that the intron-containing genes are expressed more efficiently in human cells than the intronless version of the same gene (Wiegand et al., 2003). It has also been reported that removal of an intron does not enhance gene expression if the formation of the exon-junction complex (EJC) on the spliced mRNA is blocked. In plants, it has been demonstrated that most of the 5′ first introns increase gene expression. In mammals, it has been reported that increased expression of a gene observed is due to deposition of an exon junction complex (EJC) after splicing of the introns from mature RNA. This may also be a possible mechanism for how introns function in plants; however, this has not been proven.

The term “intron” includes, but is not limited to, introns identified as introns by annotations generated in gene models. However, any sequence identified as an intron, whether it is identified through computational or experimental, means can be evaluated using this technique. There is no dependence on and/or requirement for a particular method of gene model construction and any method can be used. Thus, the free energy value per base pair (or free energy value) and/or the IMEter score can be determined for any non-coding sequence within a eukaryotic gene that has been identified as being an intron and/or that functions as an intron. Accordingly, an intron of this invention can include introns identified as first introns, second introns, or third introns, and the like.

The terms “minimum free energy” (MFE), “free energy,” and “Gibbs free energy” are used herein interchangeably. The Gibbs free energy is a thermodynamic quantity that is the difference between the enthalpy and the product of the absolute temperature and the entropy of a system. Gibbs free energy is the capacity of a system to do non-mechanical work and ΔG measures the non-mechanical work done on it. (Perrot, Pierre. A to Z of Thermodynamics. Oxford University Press (1998)). The Gibbs free energy is defined as G=H−TS, where H is the enthalpy, T is temperature and S is the entropy (H=U+pV, where p is the pressure and V is the volume).

It is generally considered that all systems strive to achieve a minimum free energy. Thus, when the change in Gibbs free energy, ΔG, is negative then a reaction is favored and energy is released. The amount of energy released is equal to the maximum amount of work that can be performed as a result of that particular chemical reaction. When conditions result in a change in Gibbs free energy, ΔG, that is positive then energy must be added to the system to make the reaction proceed. In isothermal, isobaric systems, Gibbs free energy is a representative measure of the competing effects of enthalpy and entropy that are involved in a thermodynamic process. Thus, Gibb free energy can be consider to be a dynamic quantity.

Accordingly, as used herein, minimum free energy (i.e., free energy, Gibbs free energy) identifies the value for the structure found by thermodynamic optimization (i.e., an implementation of the Zuker algorithm (M. Zuker and P. Stiegler, Nucleic Acids Research 9:133-148 (1981)) that has the lowest free energy value (i.e., Gibb's free energy; ΔG (kcal/mol); ΔG/length of nucleotide sequence (kcal/mol/base pair)). The Gibb's free energy of a sequence can be calculated using, for example, the RNAfold program as known by those of skill in the art. (See, e.g., Id., Hofacker et al. Monatshefte f. Chemie 125: 167-188 (1994); McCaskill J S. Biopolymers 29 (6-7):1105-19. (1990); and Hofacker et al. Bioinformatics 22 (10):1172-6 (2006)).

It is noted that the measurement of free energy can be biased by nucleotide sequence length. Longer nucleotide sequences have a greater range of free energies than short nucleotide sequences. Thus, nucleotide sequences of variable length can be compared by normalizing the free energy calculation (kcal/mol) by dividing the free energy by the sequence length (i.e., the mean free energy=ΔG/length of nucleotide sequence (kcal/mol/base pair)).

IMEter score is the value determined for a given intron using the calculator described in Rose et al. (“Promoter-Proximal Introns in Arabidopsis thaliana Are Enriched in Dispersed Signals that Enhance Gene Expression” Plant Cell 20:543-551 (2008)) and available at the “IMEter Online” website (korflab.ucdavis.edu/cgi-bin/web-imeter.pl; Farquharson et al. The Plant Cell 20(3):498 (2008)). As described in Rose et al., the IMEter score is derived using the number of occurrences of a given set of oligonucleotides. This derivation can be biased with regard to the length of the nucleotide sequence because oligonucleotides are more likely to appear at random to fit the IMEter pattern when they are longer sequences than shorter sequences. However, nucleotide sequences of variable length can be compared by normalizing the IMEter score by dividing the IMEter score by the sequence length (i.e., IMEter score/base pair (bp)).

Thus, in some embodiments, an intron that has a mean free energy value per base pair of below about −0.268 kcal/mol/bp can be selected for use in enhancing expression of a heterologous nucleotide sequence. Therefore, according to the methods of the present invention, any intron having a mean free energy value of below about −0.268 kcal/mol/bp is a candidate intron for use in enhancing the expression of a heterologous nucleotide sequence operably associated with said intron.

In other embodiments, an intron having a mean free energy value per base pair in a range from between about −0.268 kcal/mol/base pair (bp) and about −0.598 kcal/mol/bp can be selected for use in enhancing expression of a heterologous nucleotide sequence. Thus, in some embodiments, the intron of this invention has a mean free energy value per base pair of about −0.268 kcal/mol/base pair (bp), about −0.269 kcal/mol/bp, about −0.270 kcal/mol/bp, about −0.272 kcal/mol/bp, about −0.274 kcal/mol/bp, about −0.276 kcal/mol/bp, about −0.278 kcal/mol/bp, about −0.280 kcal/mol/bp, about −0.282 kcal/mol/bp, about −0.284 kcal/mol/bp, about −0.286 kcal/mol/bp, about −0.288 kcal/mol/bp, about −0.290 kcal/mol/bp, about −0.292 kcal/mol/bp, about −0.294 kcal/mol/bp, about −0.296 kcal/mol/bp, about −0.298 kcal/mol/bp, about −0.300 kcal/mol/bp, about −0.302 kcal/mol/bp, about −0.304 kcal/mol/bp, about −0.306 kcal/mol/bp, about −0.308 kcal/mol/bp, about −0.310 kcal/mol/bp, about −0.315 kcal/mol/bp, about −0.320 kcal/mol/bp, about −0.325 kcal/mol/bp, about −0.330 kcal/mol/bp, about −0.335 kcal/mol/bp, about −0.340 kcal/mol/bp, about −0.345 kcal/mol/bp, about −0.350 kcal/mol/bp, about −0.355 kcal/mol/bp, about −0.360 kcal/mol/bp, about −0.365 kcal/mol/bp, about −0.370 kcal/mol/bp, about −0.375 kcal/mol/bp, about −0.380 kcal/mol/bp, about −0.385 kcal/mol/bp, about −0.390 kcal/mol/bp, about −0.395 kcal/mol/bp, about −0.400 kcal/mol/bp, about −0.405 kcal/mol/bp, about −0.410 kcal/mol/bp, about −0.415 kcal/mol/bp, about −0.420 kcal/mol/bp, about −0.425 kcal/mol/bp, about −0.430 kcal/mol/bp, about −0.435 kcal/mol/bp, about −0.440 kcal/mol/bp, about −0.445 kcal/mol/bp, about −0.450 kcal/mol/bp, about −0.455 kcal/mol/bp, about −0.460 kcal/mol/bp, about −0.465 kcal/mol/bp, about −0.470 kcal/mol/bp, about −0.475 kcal/mol/bp, about −0.480 kcal/mol/bp, about −0.485 kcal/mol/bp, about −0.490 kcal/mol/bp, about −0.495 kcal/mol/bp, about −0.500 kcal/mol/bp, about −0.505 kcal/mol/bp, about −0.510 kcal/mol/bp, about −0.515 kcal/mol/bp, about −0.520 kcal/mol/bp, about −0.525 kcal/mol/bp, about −0.530 kcal/mol/bp, about −0.535 kcal/mol/bp, about −0.540 kcal/mol/bp, about −0.545 kcal/mol/bp, about −0.550 kcal/mol/bp, about −0.555 kcal/mol/bp, about −0.560 kcal/mol/bp, about −0.565 kcal/mol/bp, about −0.570 kcal/mol/bp, about −0.575 kcal/mol/bp, about −0.580 kcal/mol/bp, about −0.585 kcal/mol/bp, about −0.590 kcal/mol/bp, about −0.591 kcal/mol/bp, about −0.592 kcal/mol/bp, about −0.593 kcal/mol/bp, about −0.594 kcal/mol/bp, about −0.595 kcal/mol/bp, about −0.596 kcal/mol/bp, about −0.597 kcal/mol/bp, or about −0.598 kcal/mol/bp, and the like.

In further embodiments, an intron having a mean free energy value per base pair of below about −0.268 kcal/mol/bp and a mean IMEter score of at least about −0.034/bp can be selected for use in enhancing expression of a nucleotide sequence (e.g., a heterologous nucleotide sequence). Thus, in some embodiments, any intron having a mean free energy value of below about −0.268 kcal/mol/bp and a mean IMEter score of a least about −0.033/bp can be an intron for use in enhancing the expression of a heterologous nucleotide sequence. In still further embodiments, the selected intron has a mean free energy value per base pair in a range from between about −0.268 kcal/mol/base pair (bp) and −0.598 kcal/mol/bp, as described herein and a mean IMEter score in a range from between about −0.034 to 0.925/bp, as described herein.

Thus, in some embodiments, the selected intron has a mean IMEter score of about −0.034/bp, about −0.033/bp, about −0.032/bp, about −0.031/bp, about −0.030/bp, about −0.029/bp, about −0.028/bp, about −0.027/bp, about −0.026/bp, about −0.025/bp, about −0.024/bp, about −0.023/bp, about −0.022/bp, about −0.021/bp, about −0.020/bp, about −0.019/bp, about −0.018/bp, about −0.017/bp, about −0.016/bp, about −0.015/bp, about −0.014/bp, about −0.013/bp, about −0.012/bp, about −0.011/bp, about −0.010/bp, about −0.009/bp, about −0.008/bp, about −0.007/bp, about −0.006/bp, about −0.005/bp, about −0.004/bp, about −0.003/bp, about −0.002/bp, about −0.001/bp, about 0.0/bp, about 0.001/bp, about 0.002/bp, about 0.003/bp, about 0.004/bp, about 0.005/bp, about 0.006/bp, about 0.007/bp, about 0.008/bp, about 0.009/bp, about 0.010/bp, about 0.011/bp, about 0.012/bp, about 0.013/bp, about 0.014/bp, about 0.015/bp, about 0.016/bp, about 0.017/bp, about 0.018/bp, about 0.019/bp, about 0.020/bp, about 0.021/bp, about 0.022/bp, about 0.023/bp, about 0.024/bp, about 0.025/bp, about 0.026/bp, about 0.027/bp, about 0.028/bp, about 0.029/bp, about 0.030/bp, about 0.031/bp, about 0.032/bp, about 0.033/bp, about 0.034/bp, about 0.035/bp, about 0.036/bp, about 0.037/bp, about 0.038/bp, about 0.039/bp, about 0.040/bp, about 0.041/bp, about 0.042/bp, about 0.043/bp, about 0.044/bp, about 0.045/bp, about 0.046/bp, about 0.047/bp, about 0.048/bp, about 0.049/bp, about 0.050/bp, about 0.051/bp, about 0.052/bp, about 0.053/bp, about 0.054/bp, about 0.055/bp, about 0.056/bp, about 0.057/bp, about 0.058/bp, about 0.059/bp, about 0.060/bp, about 0.061/bp, about 0.062/bp, about 0.063/bp, about 0.064/bp, about 0.065/bp, about 0.066/bp, about 0.067/bp, about 0.068/bp, about 0.069/bp, about 0.070/bp, about 0.071/bp, about 0.072/bp, about 0.073/bp, about 0.074/bp, about 0.075/bp, about 0.076/bp, about 0.077/bp, about 0.078/bp, about 0.079/bp, about 0.080/bp, about 0.081/bp, about 0.082/bp, about 0.083/bp, about 0.084/bp, about 0.085/bp, about 0.086/bp, about 0.087/bp, about 0.088/bp, about 0.089/bp, about 0.090/bp, about 0.091/bp, about 0.092/bp, about 0.093/bp, about 0.094/bp, about 0.095/bp, about 0.096/bp, about 0.097/bp, about 0.098/bp, about 0.099/bp, about 0.100/bp, about 0.105/bp, about 0.110/bp, about 0.115/bp, about 0.120/bp, about 0.125/bp, about 0.130/bp, about 0.135/bp, about 0.140/bp, about 0.145/bp, about 0.150/bp, about 0.155/bp, about 0.160/bp, about 0.165/bp, about 0.170/bp, about 0.175/bp, about 0.180/bp, about 0.185/bp, about 0.190/bp, about 0.195/bp, about 0.200/bp, about 0.205/bp, about 0.210/bp, about 0.215/bp, about 0.220/bp, about 0.225/bp, about 0.230/bp, about 0.235/bp, about 0.240/bp, about 0.245/bp, about 0.250/bp, about 0.255/bp, about 0.260/bp, about 0.265/bp, about 0.270/bp, about 0.275/bp, about 0.280/bp, about 0.285/bp, about 0.290/bp, about 0.295/bp, about 0.300/bp, about 0.305/bp, about 0.310/bp, about 0.315/bp, about 0.320/bp, about 0.325/bp, about 0.330/bp, about 0.335/bp, about 0.340/bp, about 0.345/bp, about 0.350/bp, about 0.355/bp, about 0.360/bp, about 0.365/bp, about 0.370/bp, about 0.375/bp, about 0.380/bp, about 0.385/bp, about 0.390/bp, about 0.395/bp, about 0.400/bp, about 0.405/bp, about 0.410/bp, about 0.415/bp, about 0.420/bp, about 0.425/bp, about 0.430/bp, about 0.435/bp, about 0.440/bp, about 0.445/bp, about 0.450/bp, about 0.455/bp, about 0.460/bp, about 0.465/bp, about 0.470/bp, about 0.475/bp, about 0.480/bp, about 0.485/bp, about 0.490/bp, about 0.495/bp, about 0.500/bp, about 0.505/bp, about 0.510/bp, about 0.515/bp, about 0.520/bp, about 0.525/bp, about 0.530/bp, about 0.535/bp, about 0.540/bp, about 0.545/bp, about 0.550/bp, about 0.555/bp, about 0.560/bp, about 0.565/bp, about 0.570/bp, about 0.575/bp, about 0.580/bp, about 0.585/bp, about 0.590/bp, about 0.595/bp, about 0.600/bp, about 0.605/bp, about 0.610/bp, about 0.615/bp, about 0.620/bp, about 0.625/bp, about 0.630/bp, about 0.635/bp, about 0.640/bp, about 0.645/bp, about 0.650/bp, about 0.655/bp, about 0.660/bp, about 0.665/bp, about 0.670/bp, about 0.675/bp, about 0.680/bp, about 0.685/bp, about 0.690/bp, about 0.695/bp, about 0.700/bp, about 0.705/bp, about 0.710/bp, about 0.715/bp, about 0.720/bp, about 0.725/bp, about 0.730/bp, about 0.735/bp, about 0.740/bp, about 0.745/bp, about 0.750/bp, about 0.755/bp, about 0.760/bp, about 0.765/bp, about 0.770/bp, about 0.775/bp, about 0.780/bp, about 0.785/bp, about 0.790/bp, about 0.795/bp, about 0.800/bp, about 0.805/bp, about 0.810/bp, about 0.815/bp, about 0.820/bp, about 0.825/bp, about 0.830/bp, about 0.835/bp, about 0.840/bp, about 0.845/bp, about 0.850/bp, about 0.855/bp, about 0.860/bp, about 0.865/bp, about 0.870/bp, about 0.875/bp, about 0.880/bp, about 0.885/bp, about 0.890/bp, about 0.895/bp, about 0.900/bp, about 0.905/bp, about 0.910/bp, about 0.915/bp, about 0.920/bp, about 0.925/bp, or about 0.930/bp, and the like.

As used herein, “a,” “an” or “the” can mean one or more than one. For example, “a” cell can mean a single cell or a multiplicity of cells.

As used herein, “and/or” refers to and encompasses any and all possible combinations of one or more of the associated listed items, as well as the lack of combinations when interpreted in the alternative (“or”).

Furthermore, the term “about” as used herein when referring to a measurable value such as an amount of a compound or agent of this invention, dose, time, percentage, temperature, thermodynamic value (e.g., free energy) and the like, is meant to encompass variations of ±20%, ±10%, ±5%, ±1%, ±0.5%, or even ±0.1% of the specified amount.

It will be understood that, although the terms “first,” “second,” etc., may be used herein to describe various elements, these elements should not be limited by these terms. These terms are only used to distinguish one element from another. Thus, a “first” element (e.g., a first intron sequence) discussed below could also be termed a “second” element (e.g., a second intron sequence) without departing from the teachings of the present invention.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, “determining” a mean free energy value or free energy value and/or a mean IMEter score or an IMEter score includes looking up the value or score in a table or other source, and/or inputting the intron nucleotide sequence into a computer program, which can provide such values or scores.

The terms “enhance,” “enhanced,” “enhancing,” “enhancement,” “increase,” “increased,” and “elevate” (and grammatical variations thereof), as used herein describe an increase in the expression of a heterologous nucleotide sequence operably associated with an intron selected by the methods of the present invention. This increase can be observed by comparing the expression of a heterologous nucleotide sequence operably associated with an intron of the present invention (e.g., an intron having a mean free energy value per base pair of below about −0.268 kcal/mol/base pair (bp) and a mean IMEter score of at least about 0.034/bp) to the expression of a heterologous nucleotide sequence not operably associated with the intron of the present invention (e.g., a control).

The terms “reduce,” “reduced,” “reducing,” “reduction,” “diminish,” and “decrease” (and grammatical variations thereof), as used herein describe a decrease or reduction in the free energy value (or free energy value per base pair) of an intron that is modified. This decrease can be observed by comparing the free energy value (or free energy value per base pair) of the intron after modification to the free energy value (or free energy value per base pair) of the intron prior to modification.

“Wild type,” or “native” nucleic acid molecule, nucleotide sequence, polypeptide or amino acid sequence refers to a naturally occurring or endogenous nucleic acid molecule, nucleotide sequence, polypeptide or amino acid sequence in its naturally occurring or endogenous form (e.g., comprising the same regulatory factors, in the same order, etc., as found in nature). Thus, a “wild type intron” is an intron that is naturally occurring in or endogenous to the organism and is operably associated with the nucleotide sequence(s) as found in the wild-type organism.

As used herein, “heterologous” refers to a nucleic acid molecule or nucleotide sequence that either originates from another species or is from the same species or organism but is modified from either its original form or the form primarily expressed in the cell. Thus, a nucleic acid molecule or nucleotide sequence derived from an organism or species different from that of the cell into which the nucleic acid molecule or nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, under the control of different regulatory sequences and/or in a different position in the genome than that found in nature.

As used herein, a “heterologous polypeptide” is a polypeptide or amino acid sequence that either originates from another species or is from the same species or organism but is modified from either its original form or the form primarily produced in the cell. Thus, a polypeptide or amino acid sequence derived from an organism or species different from that of the cell into which the polypeptide or amino acid sequence is introduced, is heterologous with respect to that cell and the cell's descendants. In addition, a heterologous polypeptide or amino acid sequence includes a polypeptide or amino acid sequence derived from and inserted into the same natural, original cell type, but which is present in a non-natural state, for example, comprising a different signal sequence than that found in nature.

A further aspect of the invention provides a method of selecting an intron for enhanced expression of a nucleotide sequence encoding a polypeptide, comprising: a) determining a free energy value of one or more introns; b) determining an IMEter score of the one or more introns of (a); and c) selecting an intron having a free energy value of below about −31.51 kcal/mol and an IMEter score of at least about 40, thereby selecting an intron for enhanced expression of the nucleotide sequence encoding the polypeptide, wherein expression of the nucleotide sequence operably associated with the selected intron is enhanced as compared to expression of a nucleotide sequence not operably associated with the selected intron having a free energy value of below about −31.51 kcal/mol and an IMEter score of at least about 40. In other embodiments, the selected intron has a free energy value in a range from about −31.51 kcal/mol and about −741.84 kcal/mol and an IMEter score in a range from about 40 to about 966.

An additional aspect of the invention provides a method of selecting an intron for enhanced expression of a heterologous nucleotide sequence encoding a polypeptide, comprising: a) determining a free energy value of one or more introns; b) determining an IMEter score of the one or more introns of (a); and c) selecting an intron having a free energy value of below about −31.51 kcal/mol and an IMEter score of at least about 40, thereby selecting an intron for enhanced expression of the heterologous nucleotide sequence encoding the polypeptide, wherein expression of the heterologous nucleotide sequence operably associated with the selected intron is enhanced as compared to expression of a nucleotide sequence not operably associated with the selected intron having a free energy value of below about −31.51 kcal/mol and an IMEter score of at least about 40. In other embodiments, the selected intron has a free energy value in a range from about −31.51 kcal/mol and about −741.84 kcal/mol and an IMEter score in a range from about 40 to about 966.

The present invention further encompasses the introns selected by the methods described herein.

Thus, in some embodiments, the selected intron has a free energy value of about −31 kcal/mol, about −32 kcal/mol, about −35 kcal/mol, about −40 kcal/mol, about −45 kcal/mol, about −50 kcal/mol, about −55 kcal/mol, about −60 kcal/mol, about −65 kcal/mol, about −70 kcal/mol, about −75 kcal/mol, about −80 kcal/mol, about −85 kcal/mol, about −90 kcal/mol, about −95 kcal/mol, about −100 kcal/mol, about −105 kcal/mol, about −110 kcal/mol, about −115 kcal/mol, about −120 kcal/mol, about −125 kcal/mol, about −135 kcal/mol, about −140 kcal/mol, about −145 kcal/mol, about −150 kcal/mol, about −155 kcal/mol, about −160 kcal/mol, about −165 kcal/mol, about −170 kcal/mol, about −175 kcal/mol, about −180 kcal/mol, about −185 kcal/mol, about −190 kcal/mol, about −195 kcal/mol, about −200 kcal/mol, about −205 kcal/mol, about −210 kcal/mol, about −215 kcal/mol, about −220 kcal/mol, about −225 kcal/mol, about −230 kcal/mol, about −235 kcal/mol, about −240 kcal/mol, about −245 kcal/mol, about −250 kcal/mol, about −255 kcal/mol, about −260 kcal/mol, about −265 kcal/mol, about −270 kcal/mol, about −275 kcal/mol, about −280 kcal/mol, about −285 kcal/mol, about −290 kcal/mol, about −295 kcal/mol, about −300 kcal/mol, about −305 kcal/mol, about −310 kcal/mol, about −315 kcal/mol, about −320 kcal/mol, about −325 kcal/mol, about −330 kcal/mol, about −335 kcal/mol, about −340 kcal/mol, about −345 kcal/mol, about −350 kcal/mol, about −355 kcal/mol, about −360 kcal/mol, about −365 kcal/mol, about −370 kcal/mol, about −375 kcal/mol, about −380 kcal/mol, about −385 kcal/mol, about −390 kcal/mol, about −395 kcal/mol, about −400 kcal/mol, about −405 kcal/mol, about −410 kcal/mol, about −415 kcal/mol, about −420 kcal/mol, about −425 kcal/mol, about −430 kcal/mol, about −435 kcal/mol, about −440 kcal/mol, about −445 kcal/mol, about −450 kcal/mol, about −455 kcal/mol, about −460 kcal/mol, about −465 kcal/mol, about −470 kcal/mol, about −475 kcal/mol, about −480 kcal/mol, about −485 kcal/mol, about −490 kcal/mol, about −495 kcal/mol, about −500 kcal/mol, about −505 kcal/mol, about −510 kcal/mol, about −515 kcal/mol, about −520 kcal/mol, about −525 kcal/mol, about −530 kcal/mol, about −535 kcal/mol, about −540 kcal/mol, about −545 kcal/mol, about −550 kcal/mol, about −555 kcal/mol, about −560 kcal/mol, about −565 kcal/mol, about −570 kcal/mol, about −575 kcal/mol, about −580 kcal/mol, about −585 kcal/mol, about −590 kcal/mol, about −595 kcal/mol, about −600 kcal/mol, about −605 kcal/mol, about −610 kcal/mol, about −615 kcal/mol, about −620 kcal/mol, about −625 kcal/mol, about −630 kcal/mol, about −635 kcal/mol, about −640 kcal/mol, about −645 kcal/mol, about −650 kcal/mol, about −655 kcal/mol, about −660 kcal/mol, about −665 kcal/mol, about −670 kcal/mol, about −675 kcal/mol, about −680 kcal/mol, about −685 kcal/mol, about −690 kcal/mol, about −695 kcal/mol, about −700 kcal/mol, about −705 kcal/mol, about −710 kcal/mol, about −715 kcal/mol, about −720 kcal/mol, about −725 kcal/mol, about −730 kcal/mol, about −735 kcal/mol, about −740 kcal/mol, or about −745 kcal/mol, and the like.

In other embodiments, the selected intron has an IMEter score of about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 590, about 595, about 600, about 605, about 610, about 615, about 620, about 625, about 630, about 635, about 640, about 645, about 650, about 655, about 660, about 665, about 670, about 675, about 680, about 685, about 690, about 695, about 700, about 705, about 710, about 715, about 720, about 725, about 730, about 735, about 740, about 745, about 750, about 755, about 760, about 765, about 770, about 775, about 780, about 785, about 790, about 795, about 800, about 805, about 810, about 815, about 820, about 825, about 830, about 835, about 840, about 845, about 850, about 855, about 860, about 865, about 870, about 875, about 880, about 885, about 890, about 895, about 900, about 905, about 910, about 915, about 920, about 925, about 930, about 935, about 940, about 945, about 950, about 955, about 960, about 965, about 970, or about 970, and the like.

As used herein, the term “polypeptide” encompasses both peptides and proteins, unless indicated otherwise.

As used herein, an “isolated” polypeptide or polypeptide fragment means a polypeptide or polypeptide fragment separated or substantially free from at least some of the other components of the naturally occurring organism, for example, the cellular components or other polypeptides or nucleic acids commonly found associated with the polypeptide.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense nucleotide sequence, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides.

Nucleic acid molecules of this invention can comprise a nucleotide sequence that can be identical in sequence to the nucleotide sequence which is naturally occurring (i.e., native or wild type) or, due to the well-characterized degeneracy of the nucleic acid code, can include alternative codons that encode the same amino acid sequence as that which is found in the naturally occurring amino acid sequence. Furthermore, nucleic acid molecules of this invention can comprise nucleotide sequences that can include codons which encode conservative substitutions of amino acids as are well known in the art, such that the biological activity of the resulting polypeptide and/or fragment is retained. A nucleic acid of this invention can be single or double stranded. Additionally, the nucleic acids of this invention can also include a nucleic acid strand that is partially complementary to a part of the nucleic acid sequence or completely complementary across the full length of the nucleic acid sequence. Nucleic acid sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA or antisense RNA. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

An “isolated” nucleic acid or “isolated” nucleotide sequence of the present invention is generally free of nucleotide sequences that flank the nucleic acid or the nucleotide sequence in the genomic DNA of the organism from which the nucleic acid or nucleotide sequence was derived (such as coding sequences present at the 5′ or 3′ ends). However, the nucleic acid of this invention can include some additional bases or moieties that do not deleteriously affect the basic structural and/or functional characteristics of the nucleic acid. “Isolated” does not mean that the preparation is technically pure (homogeneous).

Thus, an “isolated nucleic acid” is made up of a nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid encoding more than one polypeptide or peptide sequence.

The term “nucleic acid fragment” will be understood to mean a nucleotide sequence of reduced length relative to a reference nucleic acid or nucleotide sequence and comprising, consisting essentially of and/or consisting of a nucleotide sequence of contiguous nucleotides identical or almost identical (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of and/or consist of oligonucleotides having a length of at least about 5, 6, 7, 8, 9, 10, 11, 12, 13, 14, 15, 16, 17, 18, 19, 20, 21, 22, 23, 24, 25, 50, 75, 100, 150, 200, 250, 300, 350, 400, 450, 500, 750, or 1000 consecutive nucleotides of a nucleic acid or nucleotide sequence according to the invention.

The term “fragment,” as applied to a polypeptide or an amino acid sequence, will be understood to mean an amino acid sequence of reduced length relative to a reference polypeptide or amino acid sequence and comprising, consisting essentially of, and/or consisting of an amino acid sequence of contiguous amino acids identical or substantially identical to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention. A fragment of a polypeptide or protein can be produced by methods well known and routine in the art, for example, by enzymatic and/or other cleavage of naturally occurring peptides or polypeptides and/or by synthetic protocols that are well known.

A polypeptide fragment can be a biologically active fragment. A “biologically active fragment” or “active fragment” refers to a fragment that retains one or more of the biological activities of the reference polypeptide. Such fragments can be tested for biological activities according to methods described in the art, which are routine methods for testing activities of polypeptides, and/or according to any art-known and routine methods for identifying such activities. The production of and testing to identify biologically active fragments of a polypeptide would be well within the scope of one of ordinary skill in the art and would be routine. Thus, the present invention further provides biologically active fragments of a polypeptide such as a polypeptide of interest and also provides the polynucleotides encoding such biologically active polypeptide fragments.

The term “transgene” as used herein, refers to any nucleic acid molecule or nucleotide sequence used in the transformation of a plant, animal, or other organism. Thus, a transgene can be a coding sequence, a non-coding sequence, a cDNA, a gene or fragment or portion thereof, a genomic sequence, a regulatory element and the like. A “transgenic” organism, such as a transgenic plant, transgenic microorganism, or transgenic animal, is an organism into which a transgene has been delivered or introduced and the transgene can be expressed in the transgenic organism to produce a product, the presence of which can impart an effect and/or a phenotype in the organism.

The terms “complementary” or “complementarity,” as used herein, refer to the natural aligning and binding of polynucleotides under permissive salt and temperature conditions by base-pairing. For example, the sequence “A-G-T” aligns and binds to the complementary sequence “T-C-A.” Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

Different nucleic acids or proteins having homology are referred to herein as “homologues.” The term homologue includes homologous sequences from the same and other species and orthologous sequences from the same and other species. “Homology” refers to the level of similarity between two or more nucleotide sequences and/or amino acid sequences in terms of percent of positional identity (i.e., sequence similarity or identity). Homology also refers to the concept of similar functional properties among different nucleic acids, amino acids or proteins.

As used herein “sequence identity” refers to the extent to which two optimally aligned polynucleotide or amino acid sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48:1073 (1988)). More particularly, preferred computer programs for determining sequence identity include but are not limited to the Basic Local Alignment Search Tool (BLAST) programs which are publicly available from the National Center for Biotechnology Information (NCBI) at the National Library of Medicine, National Institutes of Health, Bethesda, Md. 20894; see BLAST Manual, Altschul et al., NCBI, NLM, NIH; (Altschul et al., J. Mol. Biol. 215:403-410 (1990)); version 2.0 or higher of the BLAST programs allows the introduction of gaps (deletions and insertions) into alignments; for peptide sequence, BLASTX can be used to determine sequence identity; and for polynucleotide sequence, BLASTN can be used to determine sequence identity.

Thus, the introns of this invention can be used to enhance the expression of any nucleotide sequence (endogenous or heterologous) in any eukaryote or any prokaryote and to produce a polypeptide in any eukaryotic or prokaryotic organism. Organisms of the invention include, but are not limited to, bacteria (gram-positives, chlamydeae, spirochaetes, actinobacteria, cyanobacteria, and the like) archaea, plants, insects, mammals, birds, fish, fungi, and the like.

As used herein, “plant” means any plant, and thus includes, for example, angiosperms (e.g., dicots (eudicots), monocots, magnoliides, Austrobaileyales, and the like), gymnosperms (cycads, Ginko, Gnetales, and the like), bryophytes, ferns and/or fern allies. “Plant,” as used herein, additionally includes fungi (e.g., Microsporidia, Chytridiomycota, Blastocladiomycota, Neocallimastigomycota, Glomeromycota, Ascomycota, Basidiomycota, slime molds, water molds and the like) and algae (e.g., green algae, red algae, brown algae, and the like).

Non-limiting examples of plants of the present invention include soybean, beans in general, corn, cereal crops including but not limited to barley, ryes, oats, wheat, and the like, Brassica spp., clover, cocoa, coffee, cotton, flax, maize, millet, peanut, rape/canola, rice, rye, safflower, sorghum, soybean, sugarcane, sugar beet, sunflower, sweet potato, tea, vegetables including but not limited to broccoli, brussel sprouts, cabbage, carrot, cassava, cauliflower, cucurbits, lentils, lettuce, pea, peppers, potato, radish and tomato, grasses, apples, pears, peaches, apricots, citrus, avocado, banana, coconut, pineapple and walnut, and flowers such as carnations, orchids, roses, and any combination thereof.

In some embodiments, the introns of the present invention can be used to enhance the expression of a heterologous nucleotide sequence encoding a polypeptide and to produce a heterologous polypeptide in a plant, plant part and/or plant cell. In other embodiments, the introns of the present invention can be used to enhance the expression of an endogenous nucleotide sequence (e.g., present in the genome) encoding a polypeptide and to produce a polypeptide in a plant, plant part and/or plant cell. Thus, the nucleotide sequences for the introns of the present invention can be cloned into vector that does not comprise a nucleotide sequence to be expressed, and the vector can be introduced/transformed into an organism, for example, a plant, plant part and/or plant cell to enhance the expression of an endogenous nucleotide sequence.

Accordingly, in some embodiments, the present invention provides a method of producing a polypeptide in a plant, plant part or plant cell comprising: introducing into the plant, plant part or plant cell a heterologous intron having a mean free energy value per base pair below about −0.268 kcal/mol/bp and a mean IMEter score of at least about −0.034/bp; operably associating the heterologous intron with an endogenous nucleotide sequence encoding the polypeptide under conditions whereby the endogenous nucleotide sequence is expressed in the plant, plant part or plant cell, thereby producing the polypeptide in the plant, plant part or plant cell. Other aspects of the present invention provide a method of producing a polypeptide in a plant, plant part or plant cell comprising: introducing into the plant, plant part or plant cell a heterologous intron having a free energy value of below about −31.51 kcal/mol and an IMEter score of at least about 40; and operably associating the heterologous intron with an endogenous nucleotide sequence encoding the polypeptide under conditions whereby the endogenous nucleotide sequence is expressed in the plant, plant part or plant cell, thereby producing the polypeptide in the plant, plant part or plant cell. In some embodiments, the expression of the nucleotide sequence is enhanced as compared with the expression of a nucleotide sequence encoding the heterologous polypeptide not operably associated with a heterologous intron having a mean free energy value per base pair of below about −0.268 kcal/mol/bp and a mean IMEter score of at least about −0.034/bp.

In other embodiments, the present invention provides a method of producing a heterologous polypeptide in a plant, plant part and/or plant cell, comprising: introducing into the plant, plant part and/or plant cell a heterologous nucleic acid molecule comprising: a) a heterologous intron having a mean free energy value per base pair below about −0.268 kcal/mol/bp and a mean IMEter score of at least about −0.034/bp; and b) a nucleotide sequence encoding the heterologous polypeptide, operably associated with the heterologous intron, under conditions whereby the nucleotide sequence is expressed in the plant, plant part and/or plant cell, thereby producing a heterologous polypeptide in the plant, plant part and/or plant cell. In some embodiments, the expression of the nucleotide sequence is enhanced as compared with the expression of a nucleotide sequence encoding the heterologous polypeptide not operably associated with a heterologous intron having a mean free energy value per base pair of below about −0.268 kcal/mol/bp and a mean IMEter score of at least about −0.034/bp.

A further aspect of the present invention provides a method of producing a heterologous polypeptide in a plant, plant part and/or plant cell, comprising: introducing into the plant, plant part and/or plant cell a heterologous nucleic acid molecule comprising: a) a heterologous intron having a free energy value of below about −31.51 kcal/mol and an IMEter score of at least about 40; and b) a nucleotide sequence encoding the heterologous polypeptide, operably associated with the heterologous intron, under conditions whereby the nucleotide sequence is expressed in the plant, plant part and/or plant cell, thereby producing a heterologous polypeptide in the plant, plant part and/or plant cell. In some embodiments, the expression of the nucleotide sequence is enhanced as compared with the expression of a nucleotide sequence encoding the heterologous polypeptide not operably associated with a heterologous intron having a free energy value of below about −31.51 kcal/mol and an IMEter score of at least about 40.

The present invention additionally provides a method of making a plant cell that produces a polypeptide comprising: introducing into the plant cell a heterologous intron having a mean free energy value per base pair of below about −0.268 kcal/mol/bp and a mean IMEter score of at least about −0.034/bp; and operably associating the heterologous intron with an endogenous nucleotide sequence encoding the polypeptide under conditions whereby the endogenous nucleotide sequence is expressed in the plant, plant part or plant cell, thereby producing the polypeptide in the plant, plant part or plant cell. Other aspects of the present invention provide a method of making a plant cell that produces a polypeptide comprising: introducing into the plant cell a heterologous intron having a free energy value of below about −31.51 kcal/mol and an IMEter score of at least about 40; and operably associating the heterologous intron with an endogenous nucleotide sequence encoding the polypeptide under conditions whereby the endogenous nucleotide sequence is expressed in the plant, plant part or plant cell, thereby producing the polypeptide in the plant, plant part or plant cell. In some embodiments, the expression of the heterologous nucleotide sequence in the plant cell is enhanced as compared with the expression of a nucleotide sequence encoding the heterologous polypeptide not operably associated with a heterologous intron having a mean free energy value per base pair of below about −0.268 kcal/mol/bp and a mean IMEter score of at least about −0.034/bp.

The present invention additionally provides a method of making a plant cell that expresses a heterologous nucleotide sequence and/or produces a heterologous polypeptide. Thus, in one embodiment, the present invention provides a method of making a plant cell that expresses a heterologous nucleotide sequence and/or produces a heterologous polypeptide comprising: introducing into the plant cell a heterologous nucleic acid molecule comprising: a) a heterologous intron having a free energy value per base pair of below about −0.268 kcal/mol/bp and an IMEter score of at least about −0.034/bp; and b) a nucleotide sequence encoding the heterologous polypeptide, operably associated with the heterologous intron, under conditions whereby the nucleotide sequence is expressed in the plant cell, thereby making a plant cell that expresses a heterologous nucleotide sequence and/or produces a heterologous polypeptide. In some embodiments, the expression of the heterologous nucleotide sequence in the plant cell is enhanced as compared with the expression of a nucleotide sequence encoding the heterologous polypeptide not operably associated with a heterologous intron having a mean free energy value per base pair of below about −0.268 kcal/mol/bp and a mean IMEter score of at least about −0.034/bp.

The present invention further provides a method of making a plant cell that expresses a heterologous nucleotide sequence and/or produces a heterologous polypeptide comprising: introducing into the plant cell a heterologous nucleic acid molecule comprising: a) a heterologous intron having a free energy value of below about −31.51 kcal/mol and an IMEter score of at least about 40; and b) a nucleotide sequence encoding the heterologous polypeptide, operably associated with the heterologous intron, under conditions whereby the nucleotide sequence is expressed in the plant cell, thereby making a plant cell that expresses a heterologous nucleotide sequence and/or produces a heterologous polypeptide. In some embodiments, the expression of the nucleotide sequence in the plant cell is enhanced as compared with the expression of a heterologous nucleotide sequence encoding the heterologous polypeptide not operably associated with a heterologous intron having a free energy value of below about −31.51 kcal/mol and an IMEter score of at least about 40.

In some embodiments, a heterologous intron of the present invention, in addition to comprising a free energy value per base pair (or free energy value) and an IMEter score as described herein, further comprises, consists essentially of, or consists of a length of at least about 50 nucleotides.

As used herein, the term “plant part” includes but is not limited to embryos, pollen, ovules, seeds, leaves, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. Further, as used herein, “plant cell” refers to a structural and physiological unit of the plant, which comprises a cell wall and also may refer to a protoplast. A plant cell of the present invention can be in the form of an isolated single cell or can be a cultured cell or can be a part of a higher-organized unit such as, for example, a plant tissue or a plant organ.

An “isolated cell” refers to a cell that is at least partially separated from other components with which it is normally associated in its natural state. For example, an isolated cell can be a cell in culture medium.

“Introducing,” in the context of a nucleotide sequence of this invention (e.g., an intron), means contacting the nucleotide sequence with the plant, plant part, and/or plant cell in such a manner that the nucleotide sequence gains access to the interior of a cell. Where more than one nucleotide sequence is to be introduced, these nucleotide sequences can be assembled as part of a single polynucleotide or nucleic acid construct, or as separate polynucleotide or nucleic acid constructs, and can be located on the same or different constructs (e.g., transformation vectors). Accordingly, these polynucleotides can be introduced into plant cells in a single transformation event, in separate transformation events, and/or, e.g., as part of a breeding protocol. Thus, the term “transformation” as used herein refers to the introduction of a heterologous nucleic acid molecule into a cell. Transformation of a cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear and the plastid genome, and therefore includes integration of the nucleic acid molecule into, for example, the chloroplast genome and/or the nuclear genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

A nucleic acid molecule of this invention (e.g., an intron) can be introduced into a cell by any method known to those of skill in the art. In some embodiments of the present invention, transformation of a cell comprises nuclear transformation. In other embodiments, transformation of a cell comprises plastid transformation (e.g., chloroplast transformation).

Procedures for transforming plants are well known and routine in the art and are described throughout the literature. In some embodiments of the present invention, transformation of a cell comprises nuclear transformation. In other embodiments, transformation of a cell comprises plastid transformation (e.g., chloroplast transformation). Thus, introns of the present invention can be used to transform any organism using methods know to those of skill in the art.

Procedures for transforming a wide variety of organisms, including, but not limited to, a bacteriun, a cyanobacterium, an animal, a plant, a fungus, and the like, are well known and routine in the art. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Thus, in some embodiments, the introducing into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, and/or polyethyleneglycol-mediated transformation, as well as any other electrical, chemical, physical and/or biological mechanism that results in the introduction of nucleic acid into the plant, plant part and/or cell thereof, or a combination thereof.

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hofgen & Willmitzer (1988) Nucleic Acids Res. 16:9877).

Transformation of a plant, plant part and/or plant cell by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and allows for incorporation within the interior thereof. When inert particles are utilized, a vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterial cells or a bacteriophage, each containing one or more nucleic acids sought to be introduced) also can be propelled into plant tissue.

Thus, in various embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

Likewise, the genetic properties engineered into transgenic seeds and plants, plant parts, and/or plant cells of the present invention described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleotide sequence therefore can be introduced into the plant, plant part and/or plant cell in any number of ways that are well known in the art. The methods of the invention do not depend on a particular method for introducing one or more nucleotide sequences into a plant, only that they gain access to the interior of at least one cell of the plant. Where more than one nucleotide sequence is to be introduced, they can be assembled as part of a single nucleic acid construct, or as separate nucleic acid constructs, and can be located on the same or different nucleic acid constructs. Accordingly, a nucleotide sequence can be introduced into a cell in a single transformation event, in separate transformation events, or, for example, in plants, as part of a breeding protocol.

A polypeptide of the present invention (e.g., a heterologous polypeptide) can be any polypeptide. Non-limiting examples of polypeptides of this invention that are suitable for production in plants include those resulting in agronomically important traits such as herbicide resistance, virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431. The polypeptide may also be one that results in an increase in plant vigor and/or yield (including polypeptides that allow a plant to grow at different temperatures, soil conditions and levels of sunlight and precipitation), and/or one that allows identification of a plant exhibiting a trait of interest (e.g., selectable marker gene, seed coat color, etc.).

Herbicide resistance is also sometimes referred to as herbicide tolerance. Nucleic acids conferring resistance to an herbicide that inhibits the growing point or meristem, such as an imidazalinone or a sulfonylurea can be suitable. Exemplary nucleic acids in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides encoded by nucleic acids conferring resistance to glyphosate are also suitable. See, e.g., U.S. Pat. No. 4,940,835 and U.S. Pat. No. 4,769,061. U.S. Pat. No. 5,554,798 discloses transgenic glyphosate resistant maize plants, which resistance is conferred by an altered 5-enolpyruvyl-3-phosphoshikimate (EPSP) synthase gene.

Nucleic acids for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable herbicides include those that inhibit photosynthesis, such as a triazine and a benzonitrile (nitrilase). See, U.S. Pat. No. 4,810,648. Other suitable herbicides include 2,2-dichloropropionic acid, sethoxydim, haloxyfop, imidazolinone herbicides, sulfonylurea herbicides, triazolopyrimidine herbicides, s-triazine herbicides and bromoxynil. Also suitable are genes that confer resistance to a protox enzyme, or provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Insecticidal proteins useful in the invention may be produced in an amount sufficient to control insect pests, i.e., insect controlling amounts. It is recognized that the amount of production of insecticidal protein in a plant necessary to control insects may vary depending upon the cultivar, type of insect, environmental factors and the like. Nucleic acids useful for insect or pest resistance include, for example, those that encode toxins identified in Bacillus organisms. Nucleic acids encoding Bacillus thuringiensis (Bt) toxins from several subspecies have been cloned and recombinant clones have been found to be toxic to lepidopteran, dipteran and coleopteran insect larvae (for example, various delta-endotoxin genes such as Cry1Aa, Cry1Ab, Cry1Ac, Cry1B, Cry1C, Cry1D, Cry1Ea, Cry1Fa, Cry3A, Cry9A, Cry9C and Cry9B; as well as genes encoding vegetative insecticidal proteins such as Vip1, Vip2 and Vip3). A full list of Bt toxins can be found on the worldwide web at Bacillus thuringiensis Toxin Nomenclature Database maintained by the University of Sussex (see also, Crickmore et al. (1998) Microbiol. Mol. Biol. Rev, 62:807-813).

Polypeptides that are suitable for production in plants further include those that improve or otherwise facilitate the conversion of harvested cane into a commercially useful product, including, for example, increased or altered carbohydrate content and/or distribution, improved fermentation properties, increased oil content, increased protein content, improved digestibility, and increased nutraceutical content, e.g., increased phytosterol content, increased tocopherol content, increased stanol content and/or increased vitamin content. Polypeptides of the invention also include, for example, those resulting in or contributing to a reduced content of an unwanted component in a harvested crop, e.g., phytic acid, or sugar degrading enzymes. By “resulting in” or “contributing to” is intended that the polypeptide of the invention can directly or indirectly contribute to the existence of a trait of interest (e.g., increasing cellulose degradation by the use of a heterologous cellulase enzyme).

In one embodiment, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In another embodiment, the polypeptide of the invention can be a polysaccharide degrading enzyme. Plants of this invention producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), β-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like.

Further enzymes which may be used include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this invention can be cellobiohydrolase (CBH) enzymes (EC 3.2.1.91). In one embodiment, the cellobiohydrolase enzyme can be CBH1 or CBH2.

Other enzymes (polypeptides of this invention) include, but are not limited to, hemicellulases, such as mannases and arabinofuranosidases (EC 3.2.1.55); ligninases; lipases (e.g., E.C. 3.113), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74).

In some embodiments, a polypeptide of the invention further can comprise a leader peptide. As is well known in the art, a leader peptide is a peptide that is encoded upstream (5′ end of the coding sequence) of a larger open reading frame. Any leader peptide can be used with the methods of the present invention. A leader peptide can be a peptide that is found in nature or it can be a synthetic peptide designed to function as a leader peptide.

Non-limiting examples of leader peptides include signal peptides, transit peptides, targeting peptides, signal sequences, and the like. As is well known in the art, signal/transit peptides target or transport a protein encoded by a nuclear gene to a particular organelle such as the mitochondrion, plastids (e.g., apicoplast, chromoplast, chloroplast, cyanelle, thylakoid, amyloplast, and the like), and/or microbodies (e.g., single membrane-bounded small organelles, such as peroxisomes, hydrogenosomes, glyoxysomes and/or glycosome).

Leader peptides are well known in the art and can be found in public databases such as the “Signal Peptide Website: An Information Platform for Signal Sequences and Signal Peptides.” (www.signalpeptide.de); the “Signal Peptide Database” (proline.bic.nus.edu.sg/spdb/index.html) (Choo et al., BMC Bioinformatics 6:249 (2005) (available on www.biomedcentral.com/1471-2105/6/249/abstract); ChloroP (www.cbs.dtu.dk/services/ChloroP/; predicts the presence of chloroplast transit peptides (cTP) in protein sequences and the location of potential cTP cleavage sites); LipoP (www.cbs.dtu.dk/services/LipoP/; predicts lipoproteins and signal peptides in Gram negative bacteria); MITOPROT (ihg2.helmholtz-muenchen.de/ihg/mitoprot.html; predicts mitochondrial targeting sequences); PlasMit (gecco.org.chemie.uni-frankfurt.de/plasmit/index.html; predicts mitochondrial transit peptides in Plasmodium falciparum); Predotar (urgi.versailles.inra.fr/predotar/predotar.html; predicts mitochondrial and plastid targeting sequences); PTS1 (mendel.imp.ac.at/mendeljsp/sat/pts1/PTS1predictor.jsp; predicts peroxisomal targeting signal 1 containing proteins); SignalP (www.cbs.dtu.dk/services/SignalP/; predicts the presence and location of signal peptide cleavage sites in amino acid sequences from different organisms: Gram-positive prokaryotes, Gram-negative prokaryotes, and eukaryotes. The SignalP method incorporates a prediction of cleavage sites and a signal peptide/non-signal peptide prediction based on a combination of several artificial neural networks and hidden Markov models; and TargetP (www.cbs.dtu.dk/services/TargetP/); predicts the subcellular location of eukaryotic proteins—the location assignment is based on the predicted presence of any of the N-terminal presequences: chloroplast transit peptide (cTP), mitochondrial targeting peptide (mTP) or secretory pathway signal peptide (SP)). (See also, von Heijne, G., Eur J Biochem 133 (1) 17-21 (1983); Martoglio et al. Trends Cell Biol 8 (10):410-5 (1998); Hegde et al. Trends Biochem Sci 31(10):563-71 (2006); Dultz et al. J Biol Chem 283(15):9966-76 (2008); Emanuelsson et al. Nature Protocols 2(4) 953-971 (2007); Zuegge et al. 280(1-2):19-26 (2001); Neuberger et al. J Mol Biol. 328(3):567-79 (2003); and Neuberger et al. J Mol Biol. 328(3):58′-92 (2003)).

Some embodiments of the present invention are directed to expression cassettes designed to express the nucleic acids of the present invention (e.g., a selected or modified intron). As used herein, “expression cassette” means a nucleic acid molecule having at least a control sequence operably linked to a nucleotide sequence of this invention. In this manner, for example, plant promoters in operable interaction with the nucleotide sequences for the introns of the invention are provided in expression cassettes for expression in a plant, plant part and/or plant cell. Additionally, an expression cassette as used herein means a nucleic acid molecule comprising at least an intron of the present invention, which when introduced into the organism is then operably associated with an endogenous nucleotide sequence encoding a polypeptide to be expressed/produced.

As used herein, the term “promoter” refers to a region of a nucleotide sequence (e.g., a gene or nucleic acid construct) that incorporates the necessary signals for the efficient expression of a coding sequence operably associated with the promoter. This may include sequences to which an RNA polymerase binds, but is not limited to such sequences and can include regions to which other regulatory proteins bind together with regions involved in the control of protein translation and can also include coding sequences.

In particular aspects, a “promoter” of this invention is a promoter capable of initiating transcription of a nucleotide sequence in a cell of a plant. Such promoters include those that drive expression of a nucleotide sequence constitutively, those that drive expression when induced, and those that drive expression in a tissue- or developmentally-specific manner, as these various types of promoters are known in the art.

For purposes of the invention, the regulatory regions (i.e., promoters, transcriptional regulatory regions, and translational termination regions) can be native/analogous to the plant, plant part and/or plant cell and/or the regulatory regions can be native/analogous to the other regulatory regions. Alternatively, the regulatory regions may be heterologous to the plant (and/or plant part and/or plant cell) and/or to each other (i.e., the regulatory regions). Thus, for example, a promoter can be heterologous when it is operably linked to a polynucleotide from a species different from the species from which the polynucleotide was derived. Alternatively, a promoter can also be heterologous to a selected nucleotide sequence if the promoter is from the same/analogous species from which the polynucleotide is derived, but one or both (i.e., promoter and polynucleotide) are substantially modified from their original form and/or genomic locus, or the promoter is not the native promoter for the operably linked polynucleotide.

The choice of promoters to be used depends upon several factors, including, but not limited to, cell- or tissue-specific expression, desired expression level, efficiency, inducibility and selectability. For example, where expression in a specific tissue or organ is desired, a tissue-specific promoter can be used (e.g., a root specific promoter). In contrast, where expression in response to a stimulus is desired, an inducible promoter can be used. Where continuous expression is desired throughout the cells of a plant, a constitutive promoter can be used. It is a routine matter for one of skill in the art to modulate the expression of a nucleotide sequence by appropriately selecting and positioning promoters and other regulatory regions relative to that sequence.

Therefore, in some instances, constitutive promoters can be used. Examples of constitutive promoters include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter.

Moreover, tissue-specific regulated nucleic acids and/or promoters have been reported in plants. Thus, in some embodiments, tissue specific promoters can be used. Some reported tissue-specific nucleic acids include those encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Thus, the promoters associated with these tissue-specific nucleic acids can be used in the present invention.

Additional examples of tissue-specific promoters include, but are not limited to, the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO 1 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). Particularly useful for seed-specific expression is the pea vicilin promoter (Czako et al. (1992) Mol. Gen. Genet. 235:33-40; as well as U.S. Pat. No. 5,625,136).

Other useful promoters for expression in mature leaves are those that are switched on at the onset of senescence, such as the SAG promoter from Arabidopsis (Gan et al. (1995) Science 270:1986-1988). In addition, promoters functional in plastids can be used. Non-limiting examples of such promoters include the bacteriophage T3 gene 9 5′ UTR and other promoters disclosed in U.S. Pat. No. 7,579,516. Other promoters useful with the present invention include but are not limited to the S-E9 small subunit RuBP carboxylase promoter and the Kunitz trypsin inhibitor gene promoter (Kti3).

In some instances, inducible promoters can be used. Examples of inducible promoters include, but are not limited to, tetracycline repressor system promoters, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters. Other inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108.

In addition to the promoters described above, the expression cassette also can include other regulatory sequences. As used herein, the term “regulatory sequence” means a nucleotide sequence located upstream (5′ non-coding sequences), within or downstream (3′ non-coding sequences) of a coding sequence, and which influence the transcription, RNA processing or stability, and/or translation of the associated coding sequence. Regulatory sequences include, but are not limited to, enhancers, translation leader sequences, polyadenylation signal sequences and the introns of the present invention. Thus, the introns of the present invention can be used alone to enhance the expression of a heterologous nucleotide sequence or they can be used in combination with other regulatory sequences. By way of example only, the expression cassette can include translational and/or transcriptional enhancers or can include two or more translational enhancers and two or more transcriptional enhancers. The expression cassettes also can include Kozak sequence, N-terminal targeting sequences including chloroplast transit peptide and signal peptide.

A number of non-translated leader sequences derived from viruses also are known to enhance gene expression. Specifically, leader sequences from Tobacco Mosaic Virus (TMV, the “ω-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al. (1991) Virology 81:382-385). See also, Della-Cioppa et al. (1987) Plant Physiol. 84:965-968.

The expression cassette also can optionally include a transcriptional and/or translational termination region (i.e., termination region) that is functional in plants. A variety of transcriptional terminators are available for use in expression cassettes and are responsible for the termination of transcription beyond the transgene and correct mRNA polyadenylation. The termination region may be native to the transcriptional initiation region, may be native to the operably linked nucleotide sequence of interest, may be native to the plant, and/or may be derived from another source (i.e., foreign or heterologous to the promoter, the nucleotide sequence of interest, the plant to be transformed, or any combination thereof). Appropriate transcriptional terminators include, but are not limited to, the CAMV 35S terminator, the tml terminator, the nopaline synthase terminator and the pea rbcs E9 terminator. These can be used in both monocotyledons and dicotyledons. In addition, a coding sequence's native transcription terminator can be used.

A signal sequence can be operably linked to nucleic acid molecules comprising the introns of the present invention to direct the nucleic acid into a cellular compartment. In this manner, the expression cassette will comprise a nucleotide sequence encoding the intron operably linked to the heterologous nucleotide sequence(s) and to a nucleotide sequence for the signal sequence.

Regardless of the type of regulatory sequence(s) used, they can be operably linked to the nucleotide sequence of an intron of the invention. As used herein, “operably linked” or “operably associated” (and grammatical equivalents thereof) is intended to mean a functional linkage between two or more elements. For example, a polynucleotide of the invention that is operably linked to a regulatory sequence of the invention (e.g., a promoter, an intron) has a functional link that allows for expression of the polynucleotide. Operably linked elements may be contiguous or non-contiguous. Thus, for example, intervening untranslated, yet transcribed, sequences can be present between a promoter and a coding sequence, and the promoter sequence can still be considered “operably linked” to the coding sequence. When used to refer to the joining of two protein coding regions, by operably linked it is intended that the coding regions are in the same reading frame.

An expression cassette also can include a nucleotide sequence for a selectable marker, which can be used to select a transformed plant, plant part or plant cell. As used herein, “selectable marker” means a nucleic acid molecule that when expressed imparts a distinct phenotype to the plant, plant part or plant cell expressing the marker sequence and thus allows such transformed plants, plant parts or plant cells to be distinguished from those that do not have the marker (e.g., a plant, plant part or plant cell that is not transformed). Such a nucleic acid molecule may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleic acid encoding neo or nptII, which confers resistance to kanamycin, G418, and the like (Potrykus et al. (1985) Mol. Gen. Genet. 199:183-188); a nucleic acid encoding bar, which confers resistance to phosphinothricin; a nucleic acid encoding an altered 5-enolpyruvylshikimate-3-phosphate (EPSP) synthase, which confers resistance to glyphosate (Hinchee et al. (1988) Biotech. 6:915-922); a nucleic acid encoding a nitrilase such as bxn from Klebsiella ozaenae that confers resistance to bromoxynil (Stalker et al. (1988) Science 242:419-423); a nucleic acid encoding an altered acetolactate synthase (ALS) that confers resistance to imidazolinone, sulfonylurea or other ALS-inhibiting chemicals (EP Patent Publication No. 154204); a nucleic acid encoding a methotrexate-resistant dihydrofolate reductase (DHFR) (Thillet et al. (1988) J. Biol. Chem. 263:12500-12508); a nucleic acid encoding a dalapon dehalogenase that confers resistance to dalapon; a nucleic acid encoding a mannose-6-phosphate isomerase (also referred to as phosphomannose isomerase (PMI)) that confers an ability to metabolize mannose (U.S. Pat. Nos. 5,767,378 and 5,994,629); a nucleic acid encoding an altered anthranilate synthase that confers resistance to 5-methyl tryptophan; and/or a nucleic acid encoding hph that confers resistance to hygromycin. One of skill in the art is capable of choosing a suitable selectable marker for use in an expression cassette of this invention.

Additional selectable markers include, but are not limited to, a nucleic acid encoding β-glucuronidase or uidA (GUS) that encodes an enzyme for which various chromogenic substrates are known; an R-locus nucleic acid that encodes a product that regulates the production of anthocyanin pigments (red color) in plant tissues (Dellaporta et al., “Molecular cloning of the maize R-nj allele by transposon-tagging with Ac” pp. 263-282 In: Chromosome Structure and Function: Impact of New Concepts, 18th Stadler Genetics Symposium (Gustafson & Appels eds., Plenum Press 1988)); a nucleic acid encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleic acid encoding xylE that encodes a catechol dioxygenase (Zukowsky et al. (1983) Proc. Natl. Acad. Sci, USA 80:1101-1105); a nucleic acid encoding tyrosinase, an enzyme capable of oxidizing tyrosine to DOPA and dopaquinone, which in turn condenses to form melanin (Katz et al. (1983) J. Gen. Microbiol. 129:2703-2714); a nucleic acid encoding β-galactosidase, an enzyme for which there are chromogenic substrates; a nucleic acid encoding luciferase (lux) that allows for bioluminescence detection (Ow et al. (1986) Science 234:856-859); a nucleic acid encoding aequorin which may be employed in calcium-sensitive bioluminescence detection (Prasher et al. (1985) Biochem. Biophys. Res. Comm. 126:1259-1268); or a nucleic acid encoding green fluorescent protein (Niedz et al. (1995) Plant Cell Reports 14:403-406). One of skill in the art is capable of choosing a suitable selectable marker for use in an expression cassette of this invention.

An expression cassette of the present invention also can include nucleotide sequences encoding other desired traits. Such sequences can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, the nucleotide sequences of interest can be combined at any time and/or in any order. For example, a transgenic plant comprising one or more desired traits can be used as the target to introduce further traits by subsequent transformation. The additional nucleotide sequences can be introduced simultaneously in a co-transformation protocol with nucleotide sequences for the heterologous introns provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

The expression cassette also can include a coding sequence for one or more polypeptides for agronomic traits that primarily are of benefit to a seed company, grower or grain processor, for example, bacterial pathogen resistance, fungal resistance, herbicide resistance, insect resistance, nematode resistance and/or virus resistance. See, e.g., U.S. Pat. Nos. 5,304,730; 5,495,071; 5,569,823; 6,329,504 and 6,337,431. The trait also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various traits of interest, as well as methods for introducing these traits into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956. See also, on the World Wide Web at lifesci.sussex.ac.uk/home/Neil_Crickmore/Bt/.

A nucleic acid construct/expression cassette is made up of a minimum of one or more nucleotide sequences encoding proteins or gene products and the polynucleotide sequences which control their expression. Thus, the expression cassette at a minimum consists of three components: a promoter sequence, a coding sequence (cDNA/open reading frame) and a 3′ polyadenylation sequence. However, some expression cassettes also include an intron 5′ to the coding sequence or in the coding sequence. In some instances, where a high level of mRNA transcript or recombinant protein is desired, transcriptional and/or translational enhancers and/or a Kozak sequence may be included in the expression cassette.

An exemplary nucleic acid construct to be used in an expression cassette of this invention can include a promoter, a portion of the transition zone between the intron and the exon immediately preceding the intron (approximately 15 to 75 nucleotides of the transition zone), the selected/heterologous/modified intron, a portion of the transition zone between the intron and the exon immediately downstream of the intron (approximately 15 to 75 nucleotides of the transition zone), and a heterologous nucleotide sequence of interest to be expressed. The transition zone that is used with an intron of the present invention can be the same as that which is found associated with the native intron or can be from a different intron from the same species or from an intron from a different species or organism.

In addition to expression cassettes, numerous plant transformation vectors are well known to one of skill in the art, and the nucleotide sequences described herein can be used in connection with any such vectors. The selection of a vector will depend upon the preferred transformation technique and the target species for transformation. For certain target species, different antibiotic or herbicide selection markers may be preferred as discussed herein.

The term “vector” refers to a composition for transferring, delivering or introducing a nucleic acid (or nucleic acids) into a cell. A vector comprises a nucleic acid comprising the nucleotide sequence to be transferred, delivered or introduced. In some embodiments, a vector of this invention can be a viral vector, which can comprise, e.g., a viral capsid and/or other materials for facilitating entry of the nucleic acid into a cell and/or replication of the nucleic acid of the vector in the cell (e.g., reverse transcriptase or other enzymes which are packaged within the capsid, or as part of the capsid). The viral vector can be an infectious virus particle that delivers nucleic acid into a cell following infection of the cell by the virus particle.

In particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

A transgenic organism is also provided herein, comprising, consisting essentially of and/or consisting of one or more introns of this invention. A transgenic organism is additionally provided herein comprising a transformed cell of this invention.

In particular embodiments, a transgenic plant is also provided, comprising, consisting essentially of and/or consisting of one or more introns of this invention. A transgenic plant is additionally provided herein comprising a transformed plant cell of this invention.

Additionally provided herein is a transgenic seed, a transgenic pollen grain and a transgenic ovule of the transgenic plant of this invention. Further provided is a tissue culture of regenerable transgenic cells of the transgenic plant of this invention.

Also provided herein is a crop comprising a plurality of transgenic plants of the present invention, which are planted together in an agricultural field.

Additional aspects of the invention include methods of modifying the nucleotide sequence of an intron to increase expression of a nucleic acid molecule operably associated with said intron, comprising: mutating one or more nucleotides of the nucleotide sequence of the intron, wherein said mutation results in a decrease in the mean free energy value per base pair (to below about −0.268 kcal/mol/bp) and an increase in the mean IMEter score (to at least about −0.034/bp) of the intron as compared with the mean free energy value and mean IMEter score of the intron without said modifying, thereby increasing the expression of the nucleic acid molecule operably associated with said intron.

The present invention further provides methods of modifying the nucleotide sequence of an intron to increase expression of a nucleic acid molecule operably associated with said intron, comprising: mutating one or more nucleotides of the nucleotide sequence of the intron, wherein said mutation results in a decrease in the free energy value (to below about −31.51 kcal/mol) and an increase in the IMEter score (to at least 40) of the intron as compared with the free energy value and IMEter score of the intron without said modifying, thereby increasing the expression of the nucleic acid molecule operably associated with said intron. The present invention further encompasses the nucleotide sequence of an intron modified according to any of the methods described herein.

Any nucleotide sequence identified as an intron as described herein can be modified using the methods of the present invention. In some embodiments, the intron that is modified is an intron previously selected for enhanced expression of a nucleotide sequence using the methods of the present invention. Thus, the intron selected using the methods of the invention would be modified to further improve the ability of the intron to enhance expression of a nucleotide sequence encoding a polypeptide by further reducing the free energy and increasing the IMEter score of said intron. In other embodiments, the intron that is modified is not previously selected using the methods of the present invention, and is modified according to the methods of the present invention to improve the ability of the intron to enhance expression of a heterologous nucleotide sequence encoding a polypeptide by reducing the free energy and increasing the IMEter score of said intron.

The nucleotide sequence of an intron can be modified by mutating the nucleotides of the intron nucleotide sequence (e.g., by substitution, deletion, and/or insertion). Thus, for example, as shown in Table 1 below, one or more nucleotides in a sequence can be substituted with other nucleotides. Table 1 is exemplary for a sequence that is four nucleotides in length but the same can be done with any length nucleotide sequence comprising any number of mutations in any combination within that nucleotide sequence. The free energy value and IMEter score of the modified intron can then be determined. Those introns in which the mutation(s) results in a decrease in the mean free energy value per base pair (to below about 0.268 kcal/mol/bp) (or a decrease in the free energy value (to below about −31.51 kcal/mol)) and/or an increase in the mean IMEter score (to at least about −0.034/bp) (and/or an increase in the IMEter score (to at least about 40)) as compared with the free energy value and IMEter score of the intron without said modifying can be used in place of a naturally occurring intron sequence to increase/enhance the expression of a nucleotide sequence operably associated with the modified intron.

TABLE 1
Exemplary substitution mutations of a four nucleotide sequence.

| | A | C | G | T |
|---|---|---|---|---|
| Single Mutations | | | | |
| n = [A or C or G or T] | n | C | G | T |
| | A | n | G | T |
| | A | C | n | T |
| | A | C | G | n |
| Double Mutations | | | | |
| k = [A or C or G or T] | k | n | G | T |
| n = [A or C or G of T] | A | k | n | T |
| | A | C | k | n |
| | k | C | n | T |
| | A | k | G | n |
| | k | C | G | n |
| Triple Mutations | | | | |
| k = [A or C or G or T] | k | n | p | T |
| n = [A or C or G of T] | A | k | n | p |
| p = [A or C or G or T] | k | C | n | p |
| | k | n | G | p |

As discussed above, introns of the present invention that can be modified to comprise addition and/or deletion and/or substitution mutations are useful in this invention. Furthermore, in some embodiments, different types of mutations in any combination can be present in a single intron of the invention.

A further embodiment of the invention provides methods of generating intron mutations comprising, for example, the generation of 10,000 or more nucleotide sequences that are the same length as the intron of interest and determining the mean free energy/free energy value and/or the mean IMEter/IMEter score. Nucleotide sequences having a decrease in the mean free energy value per base pair (to below about 0.268 kcal/mol/bp) (or a decrease in the free energy value (to below about −31.51 kcal/mol)) and an increase in the mean IMEter score (to at least about −0.034/bp) (or an increase in the IMEter score (to at least about 40)) as compared with the free energy value and IMEter score of the intron without said modifying can be selected as candidate introns for use in increasing the expression of a nucleic acid molecule operably associated with the intron modified by mutation.

In other embodiments of the present invention, an automated random sampling procedure can be used to generate nucleotide sequences of the intron having various free energy values and IMEter scores (e.g., BioPerl). (Stajich et al. Genome Res 12(10):1611-8 (2002)). For example, for each position in the intron nucleotide sequence, nucleotides for that position are selected by sampling the possible nucleotides from a uniform distribution. Thus, in some embodiments and by way of example only, about ten thousand nucleotide sequences can be produced in this manner for each input intron nucleotide sequence. In other embodiments, a greater or smaller number of nucleotide sequences can be produced. The number of sequences produced will depend on a number of factors including the length of the input nucleotide sequence.

Accordingly, as described herein, the free energy values and/or IMEter scores are determined for the modified nucleotide sequences as described herein. Those nucleotide sequences having the desired free energy and/or IMEter scores are selected (e.g., a mean free energy value per base pair below about −0.268 kcal/mol/bp and/or a mean IMEter score of at least about −0.034/bp or a free energy value of below about −31.51 kcal/mol and/or an IMEter score of at least about 40), as described herein, and the nucleic acid molecules encoding the modified (mutated) nucleotide sequences are generated for use in transformation of a cell or an organism of interest.

Alternatively, nucleotide sequences can be constructed that have intron hallmarks (e.g., splice donor and acceptor sites) and the nucleotides between these sites can be modified to minimize free energy. Heuristics can be developed to assist in eliminating nucleotide sequences that are highly energetic.

The methods of the present invention additionally comprise regenerating a plant from a transformed plant cell of the present invention.

The present invention further provides transgenic plants, plant parts and crops made (e.g., produced) by the methods of the invention.

In further embodiments, the present invention provides computer-implemented methods of selecting an intron for enhanced expression of a heterologous nucleotide sequence encoding a polypeptide, comprising: a) determining a mean free energy value per base pair of one or more introns; b) determining a mean IMEter score of the one or more introns of (a); and c) selecting an intron having a mean free energy value per base pair of below about −0.268 kcal/mol/bp and a mean IMEter score of at least about −0.034/bp, thereby selecting an intron for enhanced expression of a heterologous nucleotide sequence encoding the polypeptide, wherein expression of a heterologous nucleotide sequence operably associated with the selected intron is enhanced as compared to expression of a nucleotide sequence not operably associated with an intron having a mean free energy value per base pair of below about −0.268 kcal/mol/bp and a mean IMEter score of at least about −0.034/bp. In some embodiments, the sequence data of the one or more introns is inputted into a database.

In additional embodiments, the present invention provides a computer-implemented method of selecting an intron for enhanced expression of a heterologous nucleotide sequence encoding a polypeptide, comprising: a) determining a free energy value of one or more introns; b) determining an IMEter score of the one or more introns of (a); and c) selecting an intron having a free energy value of below about −31.51 kcal/mol and an IMEter score of at least about 40, thereby selecting an intron for enhanced expression of a heterologous nucleotide sequence encoding the polypeptide, wherein expression of a heterologous nucleotide sequence operably associated with the selected intron is enhanced as compared to expression of a nucleotide sequence not operably associated with an intron having a free energy value of below about −31.51 kcal/mol and an IMEter score of at least about 40.

Embodiments according to the present invention provide a computer-implemented method of modifying the nucleotide sequence of an intron to increase expression of a nucleic acid molecule operably associated with said intron, comprising: mutating one or more nucleotides of the nucleotide sequence of the intron, wherein said mutation results in a decrease in the mean free energy value per base pair (to below about 0.268 kcal/mol/bp) and an increase in the mean IMEter score (to at least about −0.034/bp) of the intron as compared with the mean free energy value per base pair and mean IMEter score of the intron without said modifying.

In other embodiments, a computer-implemented method of modifying the nucleotide sequence of an intron is provided to increase expression of a nucleic acid molecule operably associated with said intron, the method comprising: mutating one or more nucleotides of the nucleotide sequence of the intron, wherein said mutation results in a decrease in the free energy value (to below about −31.51 kcal/mol) and an increase in the IMEter score (to at least 40) of the intron as compared with the free energy value and IMEter score of the intron without said modifying.

In some embodiments, systems and/or computer program products for carrying out the methods described herein are provided. Accordingly, the present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Thus, the present invention may be embodied in hardware and/or in software (including firmware, resident software, micro-code, etc.). Furthermore, embodiments of the present invention may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain or store the program for use by or in connection with the instruction execution system, apparatus, or device.

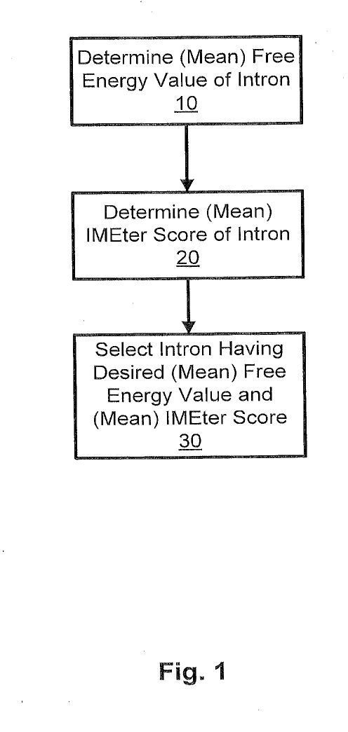

As illustrated in FIG. 1, operations according to some embodiments of the present invention include determining the free energy value for the intron nucleotide sequence (Block 10). The free energy value can be the free energy or the mean free energy value. For example, an input intron nucleotide sequence is selected and using RNAfold, the free energy value for the intron nucleotide sequence is determined. The IMEter score for the intron nucleotide sequence is determined (Block 20). The IMEter score can be the IMEter score or the mean IMEter score. The intron having the desired free energy score and IMEter score is selected (Block 30). In some embodiments, the desired mean free energy value is below about 0.268 kcal/mol/bp and the desired mean IMEter score is at least about −0.034/bp. In other embodiments, the desired free energy value is below about −31.51 kcal/mol and the desired IMEter score is at least about 40.

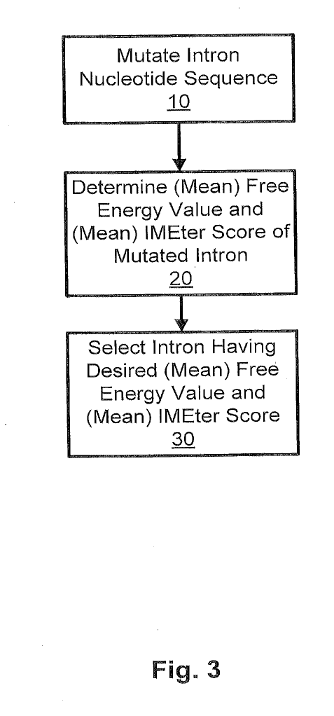

As illustrated in FIG. 3, operations according to some embodiments of the present invention include mutating intron nucleotide sequences (Block 10). For example, an input intron nucleotide sequence is selected and using a random sampling procedure, mutated intron nucleotide sequences are generated. The free energy value and the IMEter score of each intron nucleotide sequence is determined (Block 20). The free energy value can be the free energy or the mean free energy value and the IMEter score can be the IMEter score or the mean IMEter score as described for FIG. 1. The intron having the desired free energy score and IMEter score is selected (Block 30). In some embodiments, the desired mean free energy value is below about 0.268 kcal/mol/bp and the desired mean IMEter score is at least about −0.034/bp. In other embodiments, the desired free energy value is below about −31.51 kcal/mol and the desired IMEter score is at least about 40.

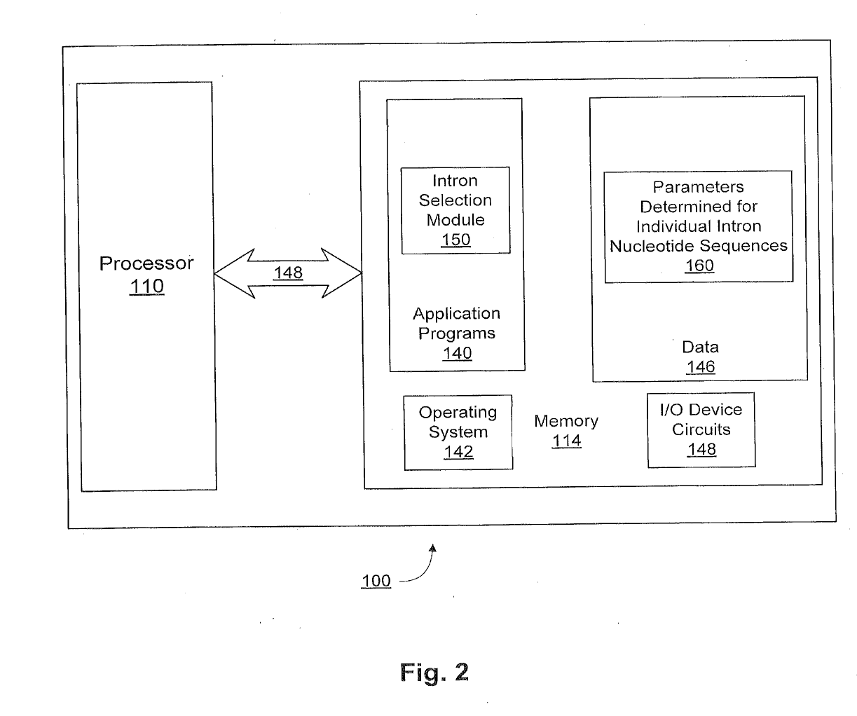

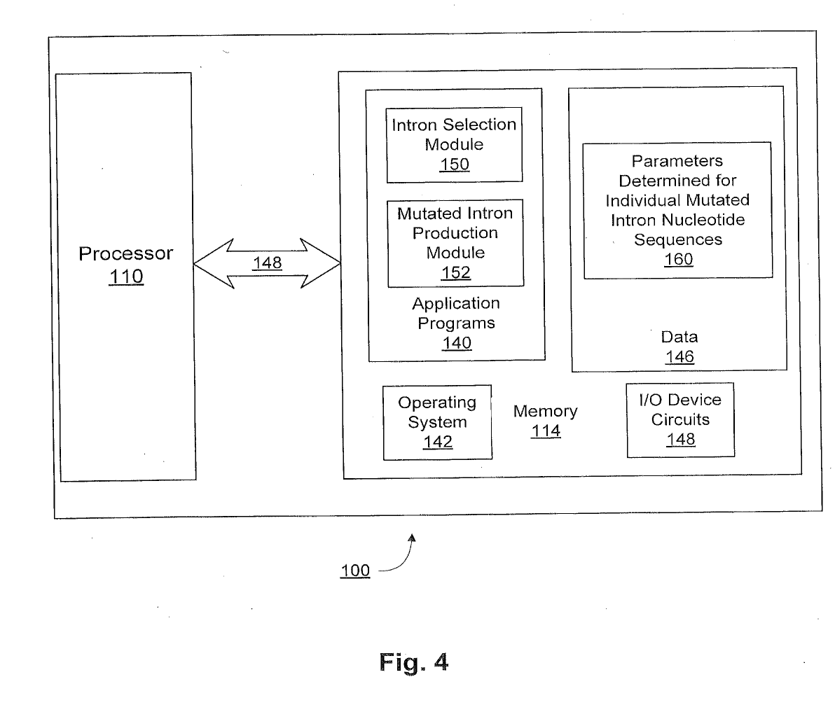

As illustrated in FIG. 2 and FIG. 4, a data processing system includes a processor 110. The processor 110 communicates with the memory 114 via an address/data bus 148. The processor 110 can be any commercially available or custom microprocessor. The memory 114 is representative of the overall hierarchy of memory devices containing the software and data used to implement the functionality of the data processing system 100. The memory 114 can include, but is not limited to, the following types of devices: cache, ROM, PROM, EPROM, EEPROM, flash memory, SRAM, and DRAM.

As shown in FIG. 2 and FIG. 4, the memory 114 may include several categories of software and data used in the data processing system: the application programs 140, the operating system 142; the input/output (I/O) device drivers 148; and the data 146. The application programs 140 can include an intron selection module 150 as in FIG. 2 and FIG. 3 and can additionally include a mutated intron production module 152 as in FIG. 4. In the intron selection module 150, introns may be selected based on the values for the parameters determined for individual introns. The data 146 may include the parameter data determined for individual intron nucleotide sequences 160. The intron selection module 150 and the mutated intron production module 152 can be configured to carry out the operations discussed in FIG. 1 and FIG. 3.

As will be appreciated by those of skill in the art, the operating system 152 shown in FIG. 2 and FIG. 4 may be any operating system suitable for use with a data processing system, such as OS/2, AIX, OS/390 or System390 from International Business Machines Corporation, Armonk, N.Y., Windows CE, Windows NT, Windows95, Windows98, Windows2000 or WindowsXP from Microsoft Corporation, Redmond, Wash., Unix or Linux or FreeBSD, Mac OS from Apple Computer, or proprietary operating systems. The I/O device drivers 148 typically include software routines accessed through the operating system 142 by the application programs 144 to communicate with devices such as I/O data port(s), data 146 and certain components of the memory 114. The application programs 140 are illustrative of the programs that implement the various features of the data processing system 100 and can include at least one application which supports operations according to embodiments of the present invention. Finally, the data 140 represents the static and dynamic data used by the application programs 140, the operating system 142, the I/O device drivers 148, and other software programs that may reside in the memory 114.

While the present invention is illustrated, for example, with reference to the intron selection module 150 being the application program in FIG. 2 and the intron selection module 150 and mutated intron production module 152 being application programs in FIG. 4, as will be appreciated by those of skill in the art, other configurations may also be utilized according to embodiments of the present invention. For example, the intron selection module 150 and/or the mutated intron production module 152 may also be incorporated into the operating system 142, the I/O device drivers 148 or other such logical division of the data processing system 100. Thus, the present invention should not be construed as limited to the configuration of FIG. 2 and/or FIG. 4, which is intended to encompass any configuration capable of carrying out the operations described herein.

The I/O data port can be used to transfer information between the data processing system 100 and another computer system or a network (e.g., the internet) or to other devices controlled by the processor. These components may be conventional components such as those used in many conventional data processing systems that may be configured in accordance with the present invention to operate as described herein. Therefore, the intron selection module 150 can be used to analyze the parameter data determined for intron nucleotide sequences 160 that have been previously collected.

The present invention will now be described with reference to the following examples. It should be appreciated that this example is for the purpose of illustrating aspects of the present invention, and does not limit the scope of the invention as defined by the claims.

### EXAMPLES

### Example 1

### Identification of Introns Nucleotide Sequences

Introns were selected from the rice genome based on their identification as introns by annotations generated in a gene model. Thus, introns were selected based upon being annotated as an “intron” within the derived or proven gene models generated for the organism that has been sequenced. In the rice genome, for example, from the TIGR rice assembly 3.0, the following sequence has been annotated as an intron.

>11667.m00001|LOC_Os01g01010.1|intron_1
(SEQ ID NO: 8)
GTAGGCTTCCTAGGTAGGGATCCCATCCCTTCGATTCCCTACTCCCTCCC
CCGATTGATTTGATTTGATTTGATTTAATTCGATTGCCTGCTTTTCAG

The free energy values and/or IMEter scores of the selected intron(s) were then measured according to methods known to those skilled in the art.

### Example 2

### Measurement of Free Energy Values and IMEter Scores for the Intron Nucleotide Sequences

The ViennaRNA package version 1.8.2 is used to calculate the free energy for each input sequence. (Hofacker et al. Monatshefte f. Chemie 125: 167-188 (1994); www.tbi.univie.ac.at/RNA/). The parameter settings for calculation were “-p0-d2,” which turns on calculation of the partition function and ensures that the partition function and the minimum free energy treat dangling end energies in the same manner. Calculation of the energies was executed on the in-house Linux cluster. In this manner, the free energy value and free energy per base pair were determined for each intron.

The IMeter Score for each generated sequence is determined using the method of Rose et al. (Plant Cell 20:543-551 (2008)) as described herein.

The free energy values and IMEter scores are provided below for seven introns in Table 2. Generally, those introns having range of IMEter scores from low to high and a range of free energy values are selected for use in testing the enhancement of expression of a heterologous nucleotide sequence encoding a polypeptide by a selected intron.

TABLE 2
Free energy values and IMEters scores for seven introns.

| | | | | Mean | Mean | | |
|---|---|---|---|---|---|---|---|
| | | IMEter | | IMEter | Free-energy | | |
| 1 | Intron length | score | Free-energy | per bp | per bp | Gene id | Protein/Known function |
| 2 | 104 | 82 | −62.2 | 0.788 | −0.598 | Os11g11410.3 | Auxin-responsive Aux/IAA gene |
| | | | | | | | family member, expressed |
| 3 | 284 | 195 | −99.3 | 0.686 | −0.349 | Os12g06540.1 | GRAS family transcription |
| | | | | | | | factor containing protein |
| 4 | 400 | 356 | −210.76 | 0.89 | −0.527 | Os07g36280.1 | F-box domain containing |
| | | | | | | | protein |
| 5 | 576 | 110 | −169.9 | 0.19 | −0.294 | Os07g31540.1 | Ubiquitin family protein, |
| | | | | | | | expressed |
| 6 | 659 | 610 | −384.7 | 0.925 | −0.584 | Os01g39350.1 | Protein kinase domain containing |
| | | | | | | | protein |
| 7 | 1016 | 315 | −310.49 | 0.31 | −0.305 | Os05g35260.2 | PB1 domain containing protein, |
| | | | | | | | expressed |
| 8 | 1714 | 966 | −741.84 | 0.563 | −0.433 | Os02g49332.1 | CSLE2-cellulose synthase-like |
| | | | | | | | family E, expressed |

The introns are evaluated both in corn and tobacco in-planta transient expression systems using a leaf specific promoter (construct 18505) and a constitutive promoter (construct 18508).

### Example 3

### Enhanced Expression of a Heterologous Nucleotide Sequence Encoding a Polypeptide

### Nucleic Acid Constructs.

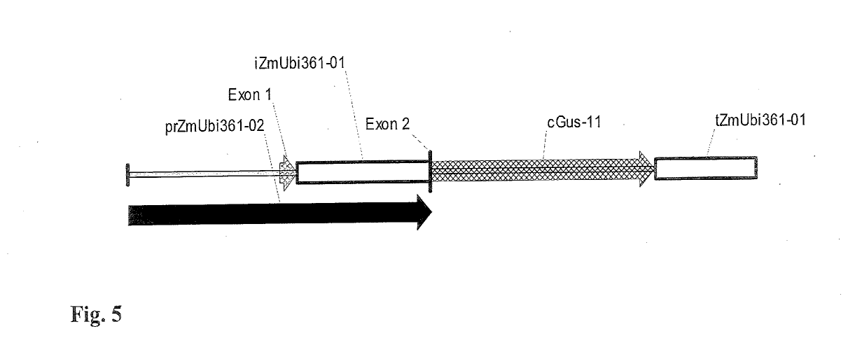

Nucleic acid constructs are prepared as shown, for example, in FIG. 5, for use in the evaluation of selected introns. The selected introns (Table 2), with a different IMEter/free energy scores, are cloned into a construct (e.g., FIG. 5). The construct has a “native” maize ubiqitin promoter from maize. The “native” promoter incorporates the features of the conventional promoter but includes the first exon, first intron, and a few bases of the second exon. The ATG of the first exon is mutated to prevent the translation so that the translation will start from the gene of interest. The gene of interest in this example is a GUS gene. The expression cassette has a native maize terminator. The native terminator also contains 3′ UTR and some downstream sequence from the maize ubiquitin gene. The first intron from the cassette is replaced with the selected introns listed in Table 2. The introns are evaluated using an in planta corn transient expression system.

A control expression cassette can be identical to the expression cassette used for enhancing the expression of a nucleotide sequence (i.e., comprising an intron of the present invention) but does not comprise an intron. Thus, for example, the expression cassette provided in FIG. 5 that is constructed without the intron, iZmUbi361-01 is a control expression cassette. Thus, a control intron can comprise a promoter, the nucleotide sequence to be expressed and a terminator.

### Transformation.

Standard methods of transformation for plants as known to those of ordinary skill in the art are used to transform plants with the nucleic acid constructs of the present invention. The introns of the present invention are then evaluated in the transformed plants using an in planta transient assay method as described below.

### In-Planta Transient Assay Method

The selected introns are cloned into an expression cassette as shown in FIG. 5 and then cloned into a standard binary vector. The constructs are transferred into Agrobacterium tumefaciens strain LBA4404 containing helper plasmid (pSBI) using a freeze-thaw method (An et al., “Binary vector” In: Gelvin S B, Schilproot R A (eds), Plant molecular biology manual. Kluwar Academic Publishers, Dordrecht, pp A3 1-19 (1988)). Preparation of Agrobacterium cultures is carried out as described by Azhakanandam et al. (Plant Mol. Biol. 63: 393-404 (2007)). In brief, the genetically modified Agrobacteria are grown overnight in 50 mL of YP medium containing 100 μM acetosyringone and 10 μM MES (pH 5.6), and subsequently are pelleted by centrifugation at 4000×g for 10 min. The pellets are resuspended in infection medium (Murashige and Skoog salts with vitamins, 2% sucrose, 500 μM MES (pH 5.6), 10 μM MgSO4, and 100 μM acetosyringone) to OD₆₀₀=0.5 and subsequently held at 28 degrees C. for 2-3 hours.

The in planta transient expression system is established using maize seedlings. Seeds are germinated under greenhouse conditions in 2.5″ pots filled with Fafard germination mix. Seedlings are kept under a 14/10 day/night cycle with a day light intensity of 2000 2.s-1 maintained with supplemental lighting. The temperature is maintained steady between 23° C.-26° C. The agroinfiltration experiment is performed with primary and secondary leaves of V2 stage seedlings (Ritchie S. W., Hanway J. J. Benson G. O. (eds): How a Corn Plant Develops: Iowa State Univ Special Report No. 48, July 2005). To make infiltration easier, the seedlings are watered 1-2 hours prior to agroinfiltration to keep the leaves turgid and the stomata open. Infiltration of individual leaves is carried out on maize seedlings using a 5 mL syringe body (BD 5 ml Syringe with Luer-Lok™ Tip, BD, Franklin Lakes, N.J. 07427, USA), by pressing the tip of the syringe against the abaxial surface of the leaf. The first and second visible leaves of V2 stage are infiltrated with: 1 ml of Agrobacterium suspension/28 seconds/leaf. Infiltrated plants are transferred and maintained under growth chamber conditions set at 25° C. with a 16/8 day/night cycle with a light intensity of 1900 μ-mol-m-2.s-1. Plant tissue is harvested after 3-4 days post infiltration for subsequent analysis.

### Stable Plant Transformation

In order to compare transient expression to expression in stable transgenic plants, the binary constructs as described above are stably transformed into maize. Maize transformation is carried out as reported (Negrotto et al., 2000; Li et al., 2003; Ishida, 1996). The transgenic events are analyzed for DNA, RNA and protein expression.

### Example 4

### Modification of the Free Energy Value and IMeter Score for the Nucleotide Sequence of an Intron

Starting with an input intron nucleotide sequence, BioPerl is used to automate a random sampling procedure that generates nucleotide sequences encoding the input intron nucleotide sequences. (Stajich et al. Genome Res 12(10):1611-8 (2002)). For each position in a nucleotide sequence, nucleotides for that position are selected by sampling the possible nucleotides from a uniform distribution. Approximately ten thousand nucleotide sequences are generated in this manner for each input nucleotide sequence. Each output nucleotide sequence is checked for length and translation to ensure the accuracy of the sampling procedure. The ViennaRNA package version 1.8.2 is used to calculate the minimum free energy for each input sequence. (Hofacker et al. Monatshefte f. Chemie 125: 167-188 (1994); www.tbi.univie.ac.at/RNA/). The parameter settings for calculation are “-p0-d2,” which turns on calculation of the partition function and ensures that the partition function and the minimum free energy treat dangling end energies in the same manner. Calculation of the energies is executed on the in-house Linux cluster. Histograms of the resulting energy distributions are plotted using R. (R Development Core Team. R: A language and environment for statistical computing. R Foundation for Statistical Computing, Vienna, Austria (2009); ISBN 3-900051-07-0; www.R-project.org). In this manner, the free energy value and free energy per base pair are determined for each intron.

In addition, the IMEter score for each modified intron nucleotide sequence is determined using the method of Rose et al. as described herein.

The modified nucleotide sequences can then be generated and used to enhance the expression of a heterologous nucleotide sequence encoding a polypeptide as described herein.

All publications, patent applications, patents and other references cited herein are incorporated by reference in their entireties for the teachings relevant to the sentence and/or paragraph in which the reference is presented.

The foregoing is illustrative of the present invention, and is not to be construed as limiting thereof. The invention is defined by the following claims, with equivalents of the claims to be included therein.

That which is claimed is:

1. A method of selecting an intron for enhanced expression of a nucleotide sequence encoding a polypeptide, comprising:
a) determining a mean free energy value per base pair (bp) of one or more introns;
b) determining a mean IMEter score of the one or more introns of (a); and
c) selecting an intron having a mean free energy value per base pair of below about −0.268 kcal/mol/bp and a mean IMEter score of at least about −0.034/bp, thereby selecting an intron for enhanced expression of the nucleotide sequence encoding the polypeptide, wherein expression of the nucleotide sequence operably associated with the selected intron is enhanced as compared to expression of a nucleotide sequence not operably associated with a heterologous intron having a mean free energy value per base pair of below about −0.268 kcal/mol/bp and a mean IMEter score of at least about −0.034/bp.

2. A method of selecting an intron for enhanced expression of a nucleotide sequence encoding a polypeptide, comprising:
a) determining a free energy value of one or more introns;
b) determining an IMEter score of the one or more introns of (a); and
c) selecting an intron having a free energy value of below about −31.51 kcal/mol and an IMEter score of at least about 40, thereby selecting an intron for enhanced expression of the nucleotide sequence encoding the polypeptide, wherein expression of the nucleotide sequence operably associated with the selected intron is enhanced as compared to expression of a nucleotide sequence not operably associated with a heterologous intron having a free energy value of below about −31.51 kcal/mol and an IMEter score of at least about 40.

3. A method of producing a heterologous polypeptide in a plant, plant part or plant cell comprising:
introducing into the plant, plant part or plant cell a heterologous nucleic acid molecule comprising:
a) a heterologous intron having a mean free energy value per base pair below about −0.268 kcal/mol/bp and a mean IMEter score of at least about −0.034/bp; and
b) a nucleotide sequence encoding the heterologous polypeptide, operably associated with the heterologous intron,
under conditions whereby the nucleotide sequence is expressed in the plant, plant part or plant cell, thereby producing a heterologous polypeptide in the plant, plant part or plant cell.

4. A method of producing a heterologous polypeptide in a plant, plant part or plant cell comprising:
introducing into the plant, plant part or plant cell a heterologous nucleic acid molecule comprising:
a) a heterologous intron having a free energy value of below about −31.51 kcal/mol and an IMEter score of at least about 40; and
b) a nucleotide sequence encoding the heterologous polypeptide, operably associated with the heterologous intron,
under conditions whereby the nucleotide sequence is expressed in the plant, plant part or plant cell, thereby producing a heterologous polypeptide in the plant, plant part or plant cell.

5. A method of making a plant cell that produces a heterologous polypeptide comprising:
introducing into the plant cell a heterologous nucleic acid molecule comprising:
a) a heterologous intron having a mean free energy value per base pair of below about −0.268 kcal/mol/bp and a mean IMEter score of at least about −0.034/bp; and
b) a nucleotide sequence encoding the heterologous polypeptide, operably associated with the heterologous intron,
under conditions whereby the nucleotide sequence is expressed in the plant cell, thereby making a plant cell that produces a heterologous polypeptide.

6. A method of making a plant cell that produces a heterologous polypeptide comprising:
introducing into the plant cell a heterologous nucleic acid molecule comprising:
a) a heterologous intron having a free energy value of below about −31.51 kcal/mol and an IMEter score of at least about 40; and
b) a nucleotide sequence encoding the heterologous polypeptide, operably associated with the heterologous intron,
under conditions whereby the nucleotide sequence is expressed in the plant cell, thereby making a plant cell that produces a heterologous polypeptide.

7. The method of claim 3, wherein the expression of the nucleotide sequence is enhanced as compared with the expression of a nucleotide sequence encoding the heterologous polypeptide not operably associated with a heterologous intron having a mean free energy value per base pair of below about −0.268 kcal/mol/bp and a mean IMEter score of at least about −0.034/bp.

8. The method of claim 4, wherein the expression of the nucleotide sequence is enhanced as compared with the expression of a nucleotide sequence encoding the heterologous polypeptide not operably associated with a heterologous intron having a free energy value of below about −31.51 kcal/mol and an IMEter score of at least about 40.

9. The method of claim 5, further comprising regenerating a plant from the plant cell.

10. A plant made by the method of claim 9.

11. A crop comprising a plurality of plants according to claim 10 planted together in an agricultural field.

12. A method of modifying the nucleotide sequence of an intron to increase expression of a nucleic acid molecule operably associated with said intron, comprising:
mutating one or more nucleotides of the nucleotide sequence of the intron, wherein said mutation results in a decrease in the mean free energy value per base pair (to below about 0.268 kcal/mol/bp) and an increase in the mean IMEter score (to at least about −0.034/bp) of the intron as compared with the free energy value and IMEter score of the intron without said modifying.

13. A method of modifying the nucleotide sequence of an intron to increase expression of a nucleic acid molecule operably associated with said intron, comprising:
mutating one or more nucleotides of the nucleotide sequence of the intron, wherein said mutation results in a decrease in the free energy value (to below about −31.51 kcal/mol) and an increase in the IMEter score (to at least about 40) of the intron as compared with the free energy value and IMEter score of the intron without said modifying.

14. The method of claim 1, further comprising modifying the selected intron by mutating one or more nucleotides of the nucleotide sequence of the selected intron, wherein said mutation results in a decrease in the mean free energy value per base pair (to below about 0.268 kcal/mol/bp) and an increase in the mean IMEter score (to at least about −0.034/bp) of the selected intron as compared with the mean free energy value and mean IMEter score of the selected intron without said modifying.

15. The method of claim 2, further comprising modifying the selected intron by mutating one or more nucleotides of the nucleotide sequence of the selected intron, wherein said mutation results in a decrease in the free energy value (to below about −31.51 kcal/mol) and an increase in the IMEter score (to at least about 40) of the selected intron as compared with the free energy value and IMEter score of the selected intron without said modifying.

16. The intron selected according to the method of claim 1.

17. The intron modified according to methods of claim 12.
